(19) United States
(12) Patent Application Publication
(10) Pub. No.: US 2023/0414119 A1
(43) Pub. Date: Dec. 28, 2023

(54) DISPLAY DEVICE AND BLOOD PRESSURE MEASUREMENT METHOD USING THE SAME

(71) Applicant: Samsung Display Co., Ltd., Yongin-Si (KR)

(72) Inventor: Chul KIM, Yongin-si (KR)

(21) Appl. No.: 18/159,945

(22) Filed: Jan. 26, 2023

(30) Foreign Application Priority Data
Jun. 27, 2022 (KR) 10-2022-0078306

(51) Int. Cl. A61B 5/022; A61B 5/021; A61B 5/024; A61B 5/026; A61B 5/00; A61B 5/1172; G06F 21/32

(52) U.S. Cl. CPC A61B 5/02225; A61B 5/02116; A61B 5/02125; A61B 5/02427; A61B 5/0261; A61B 5/6898; A61B 5/1172; G06F 21/32

(57) ABSTRACT

The display device includes: a display panel including pixels emitting light; a pressure sensor sensing a pressure; a photo-sensor sensing light; and a main processor receiving a pressure signal sensed by the pressure sensor and a first pulse wave signal sensed by the photo-sensor. The main processor calculates a first feature point corresponding to a first lowest point, a second feature point corresponding to a highest point, and a third feature point corresponding to a second lowest point in each of cycles of the first pulse wave signal, generates a peak detection signal based on an amplitude corresponding to the second feature point and the pressure signal in a first mode and calculates blood pressure information accordingly, and generates first biometric information by calculating a first ratio between a first section and a second section in a second mode and decides whether it coincides with second biometric information stored.

### CROSS-REFERENCE TO RELATED APPLICATION

This application claims priority under 35 U.S.C. § 119 to Korean Patent Application No. 10-2022-0078306, filed on Jun. 27, 2022, in the Korean Intellectual Property Office, the contents of which are herein incorporated by reference in their entirety.

### TECHNICAL FIELD

The present disclosure relates to a display device and a blood pressure measurement method using the same.

### DISCUSSION OF RELATED ART

Display devices are devices that display images on screens, and have been used not only in televisions (TVs) and monitors, but also in portable electronic devices such as, for example, mobile smartphones and tablet personal computers (PCs). Portable display devices are provided with various functions such as, for example, a camera function, a fingerprint sensor function, and the like.

Recently, as the healthcare industry has attracted attention from developers of portable devices for its increasing adoption of healthcare biometrics, methods for more conveniently acquiring biometric information regarding health have been developed. For example, there was an attempt to replace a traditional oscillometric pulse measurement device with an electronic product which can be conveniently carried. However, an electronic pulse measurement device requires an independent light source, sensor, and display in itself, and should be separately carried, which is inconvenient.

Meanwhile, biometric recognition has attracted attention because it can be used to protect personal information for each user of the display devices. The biometric recognition is an authentication method of extracting and informatizing individual's unique biometric information such as, for example, fingerprints, iris, sweat gland structures, and blood vessels, which are different for each individual. The biometric authentication method may be widely utilized in a security field because personal characteristics such as a face shape, a voice, a fingerprint, and an eyeball may not be used by theft or duplication of other people unlike keys or passwords, and there is no risk that the personal characteristics will be changed or lost. In particular, the biometric authentication method may build a safe system in terms of management because it is possible to perform posterior tracking on users. A device using such biometric recognition requires a separate biometric authentication module.

### SUMMARY

The present disclosure provides a display device capable of calculating blood pressure information in a first mode and performing biometric authentication for each user without a separate biometric authentication module in a second mode, and a blood pressure measurement method using the same.

The present disclosure is not restricted to those set forth herein. The above and other aspects of the present disclosure will become more apparent to one of ordinary skill in the art to which the present disclosure pertains by referencing the detailed description of the present disclosure given below.

According to an embodiment of the present disclosure, a display device includes a display panel including a plurality of pixels emitting light, a pressure sensor sensing a pressure applied from outside, a photo-sensor sensing light, and a main processor receiving a pressure signal sensed by the pressure sensor and a first pulse wave signal sensed by the photo-sensor, in which the main processor calculates a first feature point corresponding to a first lowest point, a second feature point corresponding to a highest point, and a third feature point corresponding to a second lowest point next to the first lowest point in each of cycles of the first pulse wave signal, generates a peak detection signal based on an amplitude corresponding to the second feature point in each of the cycles of the first pulse wave signal and the pressure signal in a first mode and calculates blood pressure information based on the peak detection signal, and generates first biometric information by calculating a first ratio between a first section from the first feature point to the second feature point and a second section from the second feature point to the third feature point in each of the cycles of the first pulse wave signal in a second mode and decides whether or not the first biometric information coincides with second biometric information stored in advance.

In an embodiment of the present disclosure, the main processor calculates a difference between a first pulse wave signal value corresponding to the second feature point and a first pulse wave signal value corresponding to the first lowest point as the amplitude.

In an embodiment of the present disclosure, a plurality of first ratios are calculated, and the first biometric information includes information on an average and a standard deviation of the first ratios of each of the cycles of the first pulse wave signal.

In an embodiment of the present disclosure, the display device further includes a memory in which the second biometric information is stored, in which the main processor is configured to store the first biometric information in the memory when the first biometric information coincides with the second biometric information stored in the memory.

In an embodiment of the present disclosure, the main processor further calculates a second ratio between an area of the first pulse wave signal in the first section and an area of the first pulse wave signal in the second section in the second mode, a plurality of second ratios are calculated, and the first biometric information further includes information on an average and a standard deviation of the second ratios.

In an embodiment of the present disclosure, the main processor further generates a second pulse wave signal by calculating a second derivative graph of the first pulse wave signal over time in the second mode, and generates the first biometric information by calculating a third ratio between a first differential value corresponding to the first feature point of the second pulse wave signal and a second differential value corresponding to the second feature point of the second pulse wave signal.

In an embodiment of the present disclosure, the main processor further calculates a peak value of the peak detection signal and a pressure value, which is referred to as PK pressure value, corresponding to the peak value of the peak detection signal in the first mode, and calculates a diastolic blood pressure lower than the PK pressure value, a systolic blood pressure higher than the PK pressure value, and a mean blood pressure according to the PK pressure value.

In an embodiment of the present disclosure, the main processor calculates the mean blood pressure as the PK pressure value corresponding to the peak value of the peak detection signal.

In an embodiment of the present disclosure, a first pressure value smaller than the PK pressure value corresponding to 60% to 80% of the peak value in the peak detection signal and a second pressure value greater than the PK pressure value are calculated, and the first pressure value is calculated as the diastolic blood pressure and the second pressure value is calculated as the systolic blood pressure.

In an embodiment of the present disclosure, each of the cycles of the first pulse wave signal includes a plurality of waveforms having different amplitudes, and when a peak value of a first waveform of the plurality of waveforms is defined as a pulse wave contraction value, a peak value of a second waveform of the plurality of waveforms is defined as a reflected pulse wave value, the pulse wave contraction value is defined as Sp, the reflected pulse wave value is defined as Rp, and a reflected pulse wave ratio is defined as RI, the pulse wave contraction value is the same as the amplitude corresponding to the second feature point in each of the cycles of the first pulse wave signal, and the main processor calculates the reflected pulse wave ratio by the following Equation 1.

$$R I = \frac{R p}{S p} \qquad Equation 1$$

In an embodiment of the present disclosure, the main processor further calculates the reflected pulse wave ratio according to each of the cycles of the first pulse wave signal in the second mode, and the first biometric information further includes the reflected pulse wave ratio.

In an embodiment of the present disclosure, the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

In an embodiment of the present disclosure, the main processor further analyzes the reflected pulse wave ratio in the first mode to detect a start point in time of the second period, calculates a third pressure value corresponding to the first pulse wave signal at the start point in time of the second period, sets the third pressure value as a diastolic blood pressure, calculates a fourth pressure value corresponding to the first pulse wave signal at a start point in time of the third period after the second period, and sets the fourth pressure value as a systolic blood pressure.

According to an embodiment of the present disclosure, a blood pressure measurement method using a display device includes calculating a first feature point corresponding to a first lowest point, a second feature point corresponding to a highest point, and a third feature point corresponding to a second lowest point next to the first feature point in each of cycles of a first pulse wave signal based on the first pulse wave signal sensed by a photo-sensor, generating a peak detection signal based on an amplitude of the second feature point in each of the cycles of the first pulse wave signal and a pressure signal sensed by a pressure sensor in a first mode, calculating blood pressure information based on the peak detection signal, generating first biometric information by calculating a first ratio between a first section from the first feature point to the second feature point and a second section from the second feature point to the third feature point in each of the cycles of the first pulse wave signal in a second mode, deciding whether or not the first biometric information coincides with second biometric information stored in advance, and displaying the blood pressure information on a display panel in the first mode and displaying a decision result for whether or not the first biometric information coincides with the second biometric information on the display panel in the second mode.

In an embodiment of the present disclosure, the amplitude of the second feature point is a difference between a first pulse wave signal value corresponding to the second feature point and a first pulse wave signal value corresponding to the first lowest point.

In an embodiment of the present disclosure, the first biometric information includes information on an average and a standard deviation of ratios between the first section and the second section of each of the cycles of the first pulse wave signal.

In an embodiment of the present disclosure, the generating of the first biometric information further includes generating a second pulse wave signal by calculating a second derivative graph of the first pulse wave signal over time, and generating the first biometric information by calculating a third ratio between a first differential value corresponding to the first feature point of the second pulse wave signal and a second differential value corresponding to the second feature point of the second pulse wave signal.

In an embodiment of the present disclosure, each of the cycles of the first pulse wave signal includes a plurality of waveforms having different amplitudes, and when a peak value of a first waveform of the plurality of waveforms is defined as a pulse wave contraction value, a peak value of a second waveform of the plurality of waveforms is defined as a reflected pulse wave value, the pulse wave contraction value is defined as Sp, the reflected pulse wave value is defined as Rp, and a reflected pulse wave ratio is defined as RI, the pulse wave contraction value is the same as the amplitude of the second feature point in each of the cycles of the first pulse wave signal, and the reflected pulse wave ratio is calculated by the following Equation 2.

$$R I = \frac{R p}{S p} \qquad Equation 2$$

In an embodiment of the present disclosure, the generating of the first biometric information further includes generating the first biometric information by calculating the reflected pulse wave ratio according to each of the cycles of the first pulse wave signal.

In an embodiment of the present disclosure, in the calculating of the blood pressure information, a peak value of the peak detection signal and a pressure value, which is referred to as PK pressure value, corresponding to the peak value of the peak detection signal are calculated in the first mode, and a diastolic blood pressure lower than the PK pressure value, a systolic blood pressure higher than the PK pressure value, and a mean blood pressure according to the PK pressure value are calculated.

According to an embodiment of the present disclosure, a display device includes a plurality of pixels emitting light, a pressure sensor sensing a pressure applied from outside, a first photo-sensor disposed in the display device to face upward and sensing light, a second photo-sensor disposed in the display device to face downward and sensing light, and a main processor receiving a pressure signal sensed by the pressure sensor, a pulse wave signal sensed by the first photo-sensor to measure a blood pressure in a first mode, and an electrocardiogram signal sensed by the second photo-sensor, in which the main processor calculates first to third feature points in each of cycles of the electrocardiogram signal, the first feature point positioned at a first peak point at a beginning of one cycle, a third feature point positioned at a second peak point at an end of the one cycle, and a second feature point positioned between the first feature point and the second feature point and at a highest point within the one cycle, calculates a fourth ratio between a first section from the first feature point to the second feature point and a second section from the second feature point to the third feature point in a second mode, calculates a plurality of fourth ratios and generates third biometric information including an average and a standard deviation of the fourth ratios of each of the cycles of the electrocardiogram signal, decides whether or not the third biometric information and fourth biometric information stored in advance in a memory are the same as each other, performs biometric authentication of a user when the third biometric information and the fourth biometric information are the same as each other, and stores the third biometric information in the memory.

With a display device and a blood pressure measurement method using the same according to an embodiment of the present disclosure, it is possible to calculate blood pressure information in a first mode and perform biometric authentication for each user without a separate biometric authentication module in a second mode by calculating a feature point for each user in a pulse wave signal sensed by a photo-sensor.

### BRIEF DESCRIPTION OF THE DRAWINGS

The above and other aspects and features of the present disclosure will become more apparent by describing in detail embodiments thereof with reference to the accompanying drawings, in which:

FIG. 1 is a plan view of a display device according to an embodiment of the present disclosure;

FIG. 2 is a plan view of the display device according to an embodiment of the present disclosure;

FIG. 3 is a block diagram illustrating the display device according to an embodiment of the present disclosure;

FIG. 4 is a plan layout view of pixels and photo-sensors of a display cell according to an embodiment of the present disclosure;

FIG. 5 is a cross-sectional view taken along line I-I′ of FIG. 4;

FIG. 6 is a block diagram illustrating a main processor according to an embodiment of the present disclosure;

FIG. 7 is a flowchart illustrating a blood pressure measurement and biometric authentication method according to an embodiment of the present disclosure;

FIG. 8 is a graph of a pressure signal indicating a pressure measurement value over time;

FIGS. 9 and 10 are graphs of a pulse wave signal indicating a pulse wave measurement value over time;

FIG. 11 is a graph illustrating one cycle of the pulse wave signal of FIG. 10;

FIG. 12 is a graph illustrating cycles of a pulse wave signal;

FIG. 13 is a flowchart illustrating a method of calculating a third ratio according to an embodiment of the present disclosure;

FIG. 14 is a graph illustrating a waveform of a pulse wave differential function;

FIG. 15 is a flowchart illustrating a method of calculating a blood pressure according to an embodiment of the present disclosure;

FIG. 16 is a graph illustrating a waveform of a peak detection signal;

FIG. 17 is a flowchart illustrating a blood pressure measurement and biometric authentication method according to an embodiment of the present disclosure;

FIG. 18 is a graph illustrating a waveform of one cycle of a pulse wave signal;

FIG. 19 is a flowchart illustrating a method of calculating a blood pressure according to an embodiment of the present disclosure;

FIG. 20 is a graph illustrating a pulse wave signal and a reflected pulse wave ratio according to an embodiment of the present disclosure;

FIG. 21 is a schematic perspective view of a display device according to an embodiment of the present disclosure;

FIG. 22 is a cross-sectional view of the display device of FIG. 21;

FIG. 23 is a flowchart illustrating a biometric authentication method according to an embodiment of the present disclosure; and

FIG. 24 is a graph illustrating a waveform of an electrocardiogram signal.

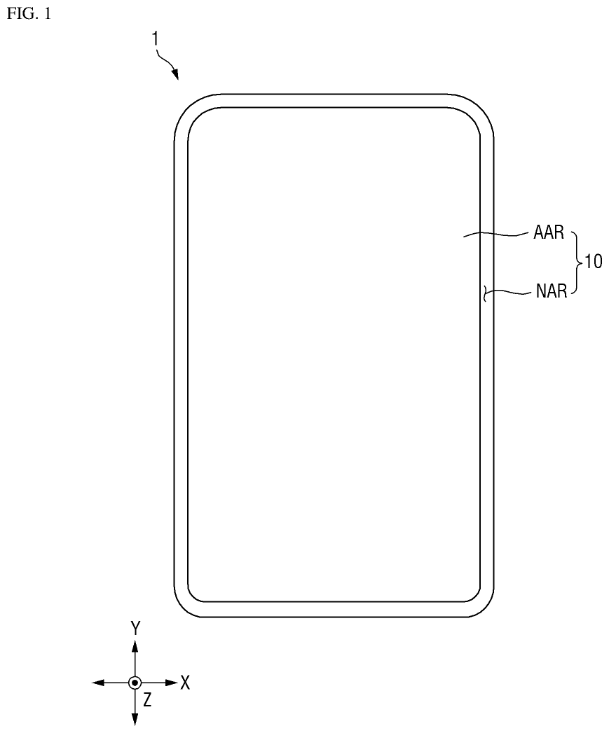

Since the drawings in FIGS. 1-24 are intended for illustrative purposes, the elements in the drawings are not necessarily drawn to scale. For example, some of the elements may be enlarged or exaggerated for clarity purpose.

### DETAILED DESCRIPTION OF THE EMBODIMENTS

The present disclosure will now be described more fully hereinafter with reference to the accompanying drawings, in which preferred embodiments of the present disclosure are shown. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art.

It will also be understood that when a layer is referred to as being “on” another layer or substrate, it can be directly on the other layer or substrate, or intervening layers may also be present. The same reference numbers indicate the same components throughout the specification.

It will be understood that, although the terms “first”, “second”, etc. may be used herein to describe various elements, these elements should not be limited by these terms. These terms are only used to distinguish one element from another element. For instance, a first element discussed below could be termed a second element without departing from the teachings of the present disclosure. Similarly, the second element could also be termed the first element.

Unless otherwise defined, all terms (including technical and scientific terms) used in this specification have the same meaning as commonly understood by one of ordinary skill in the art to which the present disclosure belongs. In addition, terms such as terms defined in commonly used dictionaries should be interpreted as having a meaning consistent with the meaning in the context of the related technology, and should not be interpreted in an ideal or overly formal sense unless explicitly defined here.

Each of the features of the various embodiments of the present disclosure may be combined or combined with each other, in part or in whole, and technically various interlocking and driving are possible. Each embodiment may be implemented independently of each other or may be implemented together in an association.

Hereinafter, specific embodiments will be described with reference to the accompanying drawings.

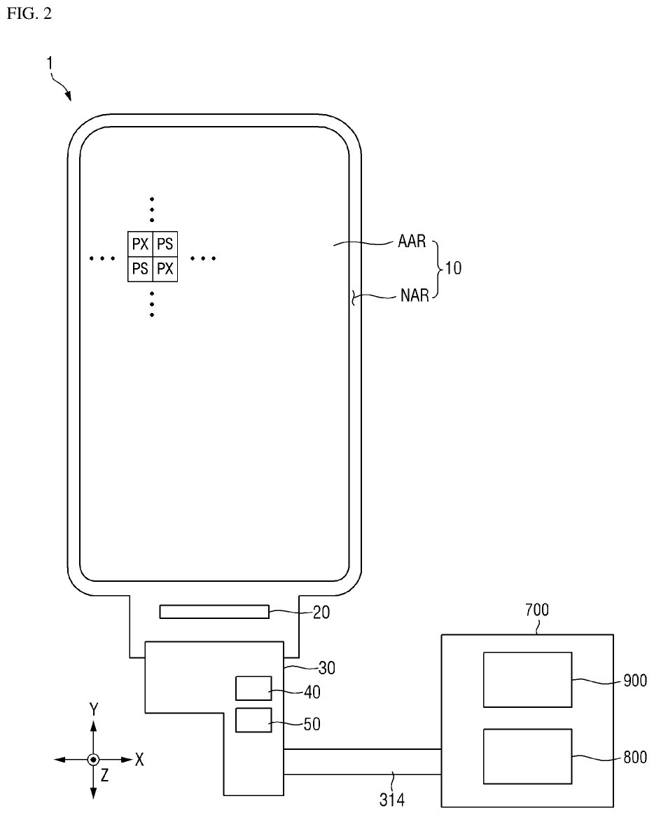

FIG. 1 is a plan view of a display device according to an embodiment of the present disclosure. FIG. 2 is a plan view of the display device according to an embodiment of the present disclosure.

Referring to FIGS. 1 and 2, a display device 1 may include various electronic devices providing a display screen. Examples of the display device 1 may include, but are not limited to, mobile phones, smartphones, tablet personal computers (PCs), mobile communication terminals, electronic notebooks, electronic books, personal digital assistants (PDAs), portable multimedia players (PMPs), navigation devices, ultra mobile PCs (UMPCs), televisions, game machines, wrist watch-type electronic devices, head-mounted displays, monitors of personal computers, laptop computers, vehicle instrument boards, digital cameras, camcorders, external billboards, electric signs, various medical devices, various inspection devices, various home appliances including display areas, such as refrigerators and washing machines, Internet of Things (IoT) devices, or the like. Representative examples of a display device 1 to be described later may include smartphones, tablet PCs, laptop computers, or the like, but are not limited thereto.

The display device 1 may include a display panel 10, a display driver 20, a circuit board a pulse wave sensing circuit 50, a pressure sensing circuit 40, a main circuit board 700, a main processor 800, and a memory 900.

The display panel 10 may include an active area AAR and a non-active area NAR.

The active area AAR includes a display area in which a screen is displayed. The active area AAR may completely overlap the display area. A plurality of pixels PX displaying an image may be disposed in the display area. Each pixel PX may include a light emitting unit emitting light.

The active area AAR further includes a light sensing area. The light sensing area is an area responding to light, and is an area configured to sense an amount, a wavelength, or the like, of incident light. The light sensing area may overlap the display area. In an embodiment of the present disclosure, the light sensing area may completely overlap the active area AAR in plan view. In this case, the light sensing area and the display area may be the same as each other. In an embodiment of the present disclosure, the light sensing area may be disposed only in a portion of the active area AAR. For example, the light sensing area may be disposed only in a limited area required for fingerprint recognition. In this case, the light sensing area may overlap a portion of the display area, but may not overlap another portion of the display area.

A plurality of photo-sensors PS responding to light may be disposed in the light sensing area. For example, the active area AAR may include a plurality of pixels PX to display an image, and a plurality of photo-sensors PS to sense incident light, for example, to be able to recognize a fingerprint.

The non-active area NAR may be disposed around the active area AAR. The display driver 20 may be disposed in the non-active area NAR, and may drive the plurality of pixels PX and/or the plurality of photo-sensors PS. The display driver 20 may output signals and voltages for driving the display panel 10. The display driver 20 may be formed as an integrated circuit (IC) and be mounted on the display panel 10. Signal lines for transferring signals between the display driver 20 and the active area AAR may be further disposed in the non-active area NAR. As another example, the display driver 20 may be mounted on the circuit board 30. An example in which the display device 1 according to an embodiment of the present disclosure includes one circuit board 30 is illustrated, but the present disclosure is not limited thereto. For example, in an embodiment of the present disclosure, the display device 1 may include a plurality of circuit boards 30 capable of being connected to the display panel 10.

The circuit board 30 may be attached to one end of the display panel 10 using an anisotropic conductive film (ACF). Lead lines of the circuit board 30 may be electrically connected to pad parts of the display panel 10. The circuit board 30 may be a flexible printed circuit board or a flexible film such as a chip on film.

The pulse wave sensing circuit 50 may be disposed on the circuit board 30. For example, the pulse wave sensing circuit 50 may be formed as an integrated circuit and be attached to an upper surface of the circuit board 30. The pulse wave sensing circuit 50 may be connected to a display layer of the display panel 10, and may sense a photocurrent generated by photocharges incident on the plurality of photo-sensors PS of the display panel 10. The pulse wave sensing circuit 50 may recognize a pulse wave of a user based on the photocurrent. A detecting driver connected to the display panel 10 may be part of the pulse wave sensing circuit 50 or may be a separate structure interacting with the pulse wave sensing circuit 50.

The pressure sensing circuit 40 may be disposed on the circuit board 30. For example, the pressure sensing circuit 40 may be formed as an integrated circuit and be attached to the upper surface of the circuit board 30. The pressure sensing circuit 40 may be connected to the display layer of the display panel 10, and may sense electrical signals by pressures applied to a plurality of pressure sensors of the display panel 10. The pressure sensing circuit 40 may generate pressure data according to a change in the electrical signal sensed by the pressure sensor, and transmit the pressure data to the main processor 800.

The main circuit board 700 may be a printed circuit board or a flexible printed circuit board.

The main circuit board 700 may include the main processor 800 and the memory 900. The main circuit board 700 may be connected to the circuit board 30 through a connection wiring pattern 314.

The main processor 800 may control all functions of the display device 1. For example, the main processor 800 may output digital video data to the display driver 20 through the circuit board 30 so that the display panel 10 displays an image. In addition, the main processor 800 may receive touch data from a touch driving circuit, decide touch coordinates of the user, and then execute an application indicated by an icon displayed on the touch coordinates of the user.

The main processor 800 may calculate a pulse wave signal PPG reflecting a blood change depending on a heartbeat according to an optical signal input from the pulse wave sensing circuit 50. In addition, the main processor 800 may calculate a touch pressure of the user according to the electrical signal input from the pressure sensing circuit 40. In addition, the main processor 800 may calculate a blood pressure of the user based on the pulse wave signal PPG and a pressure signal in a first mode. In addition, the main processor 800 may perform biometric authentication of the user based on the pulse wave signal PPG in a second mode.

The main processor 800 may be an application processor formed as an integrated circuit, a central processing unit, or a system chip.

In addition, a mobile communication module capable of transmitting and receiving wireless signals to and from at least one of a base station, an external terminal, and a server over a mobile communication network may be further mounted on the main circuit board 700. The wireless signal may include various types of data according to transmission/reception of, for example, a voice signal, a video call signal, or a text/multimedia message.

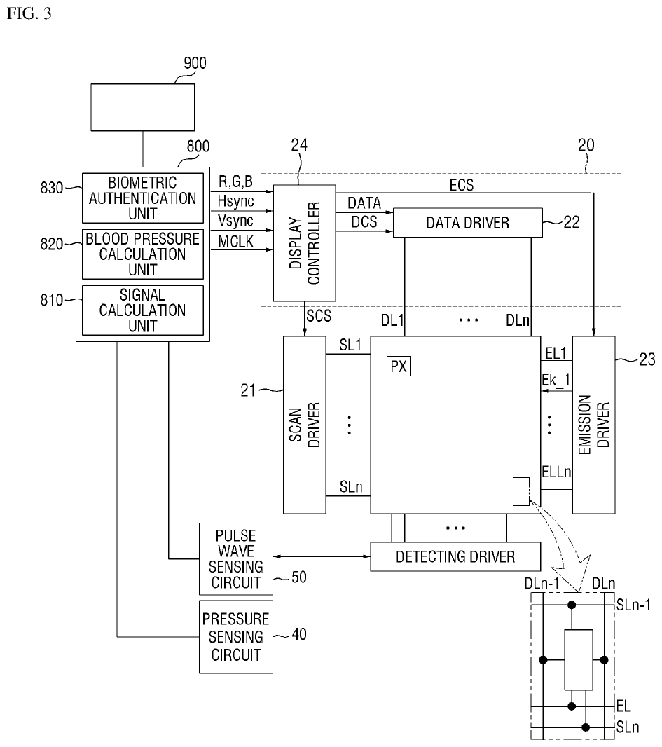

FIG. 3 is a block diagram illustrating the display device according to an embodiment of the present disclosure.

Referring to FIG. 3, the display device 1 includes the display panel 10 including the plurality of pixels PX, the display driver 20, a scan driver 21, an emission driver 23, the pulse wave sensing circuit 50, the pressure sensing circuit 40, the main processor 800, and the memory 900.

The main processor 800 may calculate the blood pressure in the first mode and perform biometric authentication for each user in the second mode. For example, in the first mode, the main processor 800 may receive electrical signals from the pulse wave sensing circuit 50 and the pressure sensing circuit 40 and calculate blood pressure information of the user. In addition, in the second mode, the main processor 800 may receive an electrical signal from the pulse wave sensing circuit 50, generate biometric authentication information of the user, and compare the biometric authentication information of the user with biometric authentication information stored in the memory 900 to perform biometric authentication of the user.

The main processor 800 includes a signal calculation unit 810, a blood pressure calculation unit 820, and a biometric authentication unit 830.

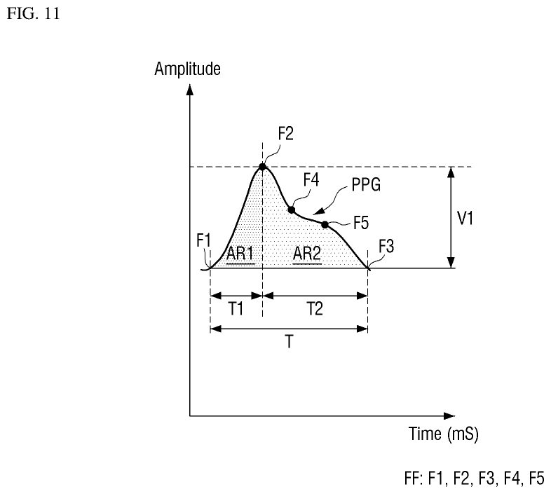

The signal calculation unit 810 may receive an optical signal from the pulse wave sensing circuit 50. In addition, the signal calculation unit 810 may receive an electrical signal from the pressure sensing circuit 40. The signal calculation unit 810 may block noise components of the received signals and calculate a feature point FF (see FIG. 11) of a pulse wave signal PPG reflecting a blood change depending on a heartbeat. The signal calculation unit 810 may output information of the feature point FF to the blood pressure calculation unit 820 and the biometric authentication unit 830.

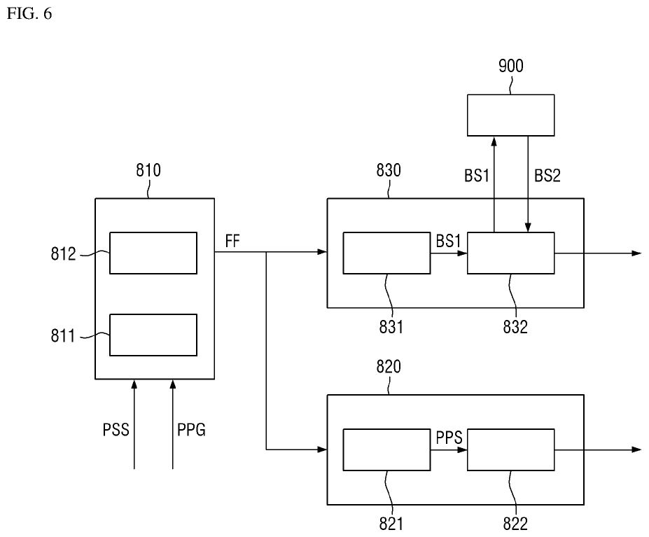

The blood pressure calculation unit 820 may receive data of the feature point FF (see FIG. 11) of the pulse wave signal PPG from the signal calculation unit 810. The blood pressure calculation unit 820 may calculate a blood pressure of the user based on the data of the feature point FF (see FIG. 11) in the first mode. To be described, the feature points FF may be defined by inflection points of a waveform formed within one heartbeat cycle T. The biometric authentication unit 830 may receive the data of the feature point FF (see FIG. 11) of the pulse wave signal PPG from the signal calculation unit 810. The biometric authentication unit 830 may perform biometric authentication by calculating biometric authentication information of the user based on the data of the feature point FF in the second mode. The signal calculation unit 810, the blood pressure calculation unit 820, and the biometric authentication unit 830 will be described later with reference to FIG. 6.

The main processor 800 drives and controls the pulse wave sensing circuit 50, the pressure sensing circuit 40, and a display controller 24. The main processor 800 may output image information to the display controller 24. For example, the main processor 800 may output image information including the calculated pulse wave signal PPG, a blood pressure measurement value, and blood pressure information to the display controller 24. For example, the display controller 24 may receive various signals such as, for example, a horizontal synchronization signal Hsync, a vertical synchronization signal Vsync, a main clock signal MCLK, and an image signal (which may include information of image colors, such as, red, green and blue R, G, B) from the main processor 800.

The display controller 24 receives an image signal supplied from the main processor 800. In addition, the display controller 24 may generate a scan control signal SCS for controlling an operation timing of the scan driver 21, an emission control signal ECS for controlling an operation timing of the emission driver 23, and a data control signal DCS for controlling an operation timing of a data driver 22. The display controller 24 may output image data DATA and the data control signal DCS to the data driver 22. The display controller 24 may output the scan control signal SCS to the scan driver 21 and output the emission control signal ECS to the emission driver 23.

The display controller 24 may be electrically connected to the display panel 10 and/or the main processor 800 through lines or may be connected to the display panel 10 and/or the main processor 800 through a communication network. In an embodiment of the present disclosure, at least a portion of the display controller 24 may be directly attached onto the display panel 10 in the form of a driving chip.

The data driver 22 may receive the image data DATA and the data control signal DCS from the display controller 24. The data driver 22 may convert the image data DATA into analog data voltages according to the data control signal DCS. The data driver 22 may output the converted analog data voltages to data lines DL in synchronization with scan signals.

The scan driver 21 may generate scan signals according to the scan control signal SCS, respectively, and sequentially output the scan signals to scan lines SL1 to SLn.

The display device 1 may further include a driving voltage, a common voltage, and a source voltage line. The source voltage line may include a driving voltage line and a common voltage line. The driving voltage may be a high potential voltage for driving light emitting elements and photoelectric conversion elements, and the common voltage may be a low potential voltage for driving the light emitting elements and the photoelectric conversion elements. That is, the driving voltage may have a higher potential than the common voltage.

A display control signal may include the scan control signal SCS, the data control signal DCS, and the emission control signal ECS. The display control signal may be output from the scan driver 21 and the data driver 22.

The emission driver 23 may generate emission signals Ek_1 according to the emission control signal ECS, and sequentially output the emission signals Ek_1 to emission lines ELL. Meanwhile, it has been illustrated that the emission driver 23 exists separately from the scan driver 21, but the present disclosure is not limited thereto, and the emission driver 23 may be included in the scan driver 21.

The data driver 22 and the display controller 24 may be included in the display driver 20 controlling an operation of the display panel 10. The data driver 22 and the display controller 24 may be formed as integrated circuits (ICs) and be mounted on the display driver 20.

Each of the plurality of pixels PX may be connected to at least one of the scan lines SL1 to SLn, any one of the data lines DL, and at least one of the emission lines ELL.

Each of the plurality of photo-sensors PS may be connected to any one of the scan lines SL1 to SLn and any one of readout lines.

A plurality of scan lines SL1 to SLn may connect the scan driver 21 to the plurality of pixels PX and the plurality of photo-sensors PS, respectively. The plurality of scan lines SL1 to SLn may provide the scan signals output from the scan driver 21 to the plurality of pixels PX, respectively.

A plurality of data lines DL may connect the data driver 22 to the plurality of pixels PX, respectively. The plurality of data lines DL may provide the image data output from the data driver 22 to the plurality of pixels PX, respectively.

A plurality of emission lines ELL may connect the emission driver 23 to the plurality of pixels PX, respectively. The plurality of emission lines ELL may provide the emission control signals output from the emission driver 23 to the plurality of pixels PX, respectively.

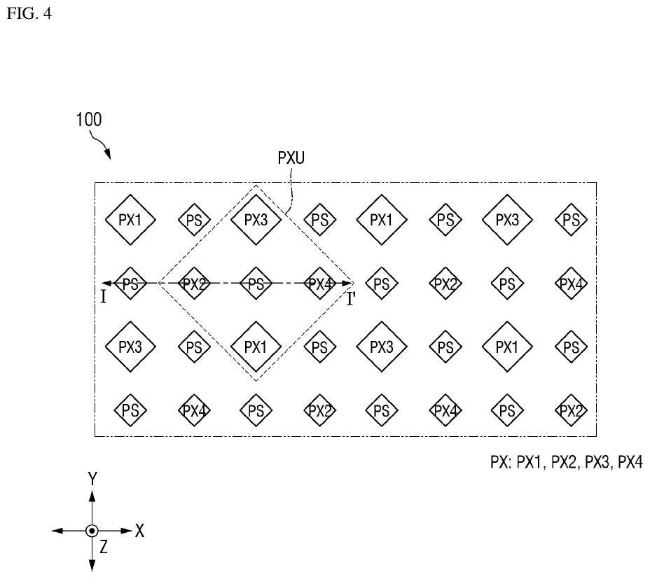

FIG. 4 is a plan layout view of pixels and photo-sensors of a display cell according to an embodiment of the present disclosure.

Referring to FIG. 4, a plurality of pixels PX and a plurality of photo-sensors PS may be repeatedly disposed in a display cell 100.

The plurality of pixels PX: PX1, PX2, PX3, and PX4 may include first pixels PX1, second pixels PX2, third pixels PX3, and fourth pixels PX4. For example, the first pixel PX1 may emit light of a red wavelength, the second pixel PX2 and the fourth pixel PX4 may emit light of a green wavelength, and the third pixel PX3 may emit light of a blue wavelength. The plurality of pixels PX may include a plurality of emission areas emitting light, respectively. The plurality of photo-sensors PS may include a plurality of light sensing areas sensing light incident thereon.

The first pixels PX1, the second pixels PX2, the third pixels PX3, and the fourth pixels PX4 and the plurality of photo-sensors PS may be alternately arranged in a first direction X and a second direction Y. The first direction X may be perpendicular to the second direction Y. In an embodiment of the present disclosure, the first pixels PX1 and the third pixels PX3 may be alternately arranged while forming a first row along the first direction X, and the second pixels PX2 and the fourth pixels PX4 may be repeatedly arranged along the first direction X in a second row adjacent to the first row. Pixels PX belonging to the first row may be staggered with pixels PX belonging to the second row in the first direction X. Arrangements of the first row and the second row may be repeated up to an n-th row. In an embodiment of the present disclosure, the first pixels PX1 and the third pixels PX3 may be alternately and repeatedly arranged while forming a first column along the second direction Y, and the second pixels PX2 and the fourth pixels PX4 may be alternately and repeatedly arranged along the second direction Y in a second column adjacent to the first column.

The photo-sensors PS may be disposed between the first pixels PX1 and the third pixels PX3 forming the first row and be disposed to be spaced apart from each other. The first pixels PX1, the photo-sensors PS, and the third pixels PX3 may be alternately arranged along the first direction X. The photo-sensors PS may be disposed between the second pixels PX2 and the fourth pixels PX4 forming the second row and be disposed to be spaced apart from each other. The second pixels PX2, the photo-sensors PS, and the fourth pixels PX4 may be alternately arranged along the first direction X. The number of photo-sensors PS in the first row may be the same as the number of photo-sensors PS in the second row. Arrangements of the first row and the second row may be repeated up to an n-th row. In an embodiment of the present disclosure, the photo-sensors PS may be disposed between the first pixels PX1 and the third pixels PX3 forming the first column and be disposed to be spaced apart from each other. Also, the photo-sensors PS may be disposed between the second pixels PX2 and the fourth pixels PX4 forming the second column and be disposed to be spaced apart from each other.

As another example, the photo-sensors PS may be disposed between the second pixels PX2 and the fourth pixels PX4 forming the second row, and may not be disposed between the first pixels PX1 and the third pixels PX3 forming the first row. That is, the photo-sensors PS may not be disposed in the first row.

Sizes of emission areas of the respective pixels PX may be different from each other. Sizes of emission areas of the second pixel PX2 and the fourth pixel PX4 may be smaller than those of emission areas of the first pixel PX1 or the third pixel PX3. It has been illustrated in FIG. 4 that the respective pixels PX have a rhombic shape, but the present disclosure is not limited thereto, and the respective pixels PX have may have, for example, a rectangular shape, an octagonal shape, a circular shape, or other polygonal shapes.

One pixel unit PXU may include one first pixel PX1, one second pixel PX2, one third pixel PX3, and one fourth pixel PX4. The pixel unit PXU refers to a group of color pixels capable of expressing a gradation.

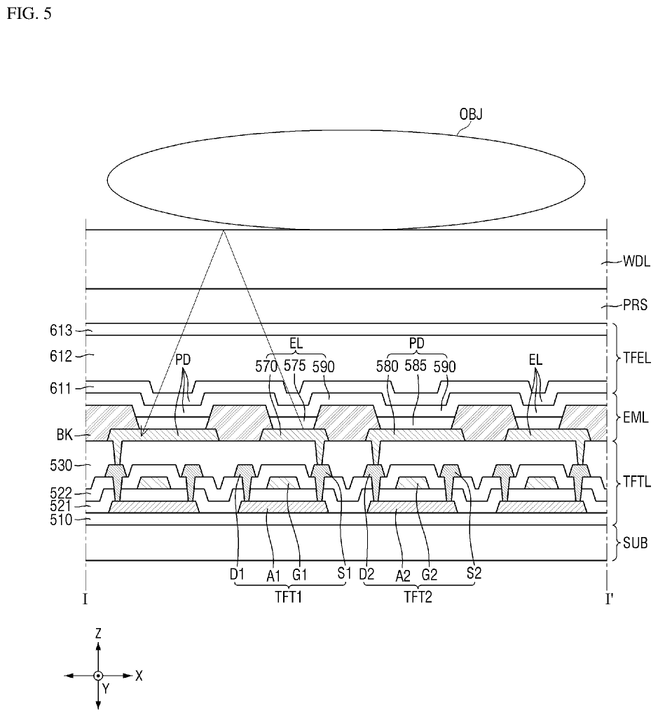

FIG. 5 is a cross-sectional view taken along line I-I′ of FIG. 4.

Referring to FIG. 5, a buffer layer 510 is disposed on a substrate SUB. The buffer layer 510 may include, for example, silicon nitride (SiNₓ), silicon oxide (SiOₓ), silicon oxynitride (SiON), or the like.

A first thin film transistor TFT1 and a second thin film transistor TFT2 may be disposed on the buffer layer 510.

A plurality of thin film transistors TFT1 and TFT2 may include, respectively, semiconductor layers A1 and A2, a gate insulating layer 521 disposed on portions of the semiconductor layers A1 and A2, gate electrodes G1 and G2 disposed on the gate insulating layer 521, an interlayer insulating layer 522 covering each of the semiconductor layers A1 and A2 and each of the gate electrodes G1 and G2, and source electrodes S1 and S2 and drain electrodes D1 and D2 disposed on the interlayer insulating layer 522.

The semiconductor layers A1 and A2 may form channels of the first thin film transistor TFT1 and the second thin film transistor TFT2, respectively. The semiconductor layers A1 and A2 may include polycrystalline silicon (p-Si). In an embodiment of the present disclosure, the semiconductor layers A1 and A2 may include single crystal silicon (sc-Si), low-temperature polycrystalline silicon (p-Si), amorphous silicon (a-Si), or an oxide semiconductor. The oxide semiconductor may include, for example, a binary compound (ABₓ), a ternary compound (ABₓC_{y}), or a quaternary compound (ABₓC_{y}D_{z}) containing indium (In), zinc (Zn), gallium (Ga), tin (Sn), titanium (Ti), aluminum (Al), hafnium (Hf), zirconium (Zr), magnesium (Mg), and the like. The semiconductor layers A1 and A2 may include channel regions, and source regions and drain regions doped with impurities, respectively.

The gate insulating layer 521 is disposed on the semiconductor layers A1 and A2. The gate insulating layer 521 electrically insulates a first gate electrode G1 and a first semiconductor layer A1 from each other, and electrically insulates a second gate electrode G2 and a second semiconductor layer A2 from each other. The gate insulating layer 521 may be made of an insulating material, for example, silicon oxide (SiOₓ), silicon nitride (SiNₓ), or metal oxide.

The first gate electrode G1 of the first thin film transistor TFT1 and the second gate electrode G2 of the second thin film transistor TFT2 are disposed on the gate insulating layer 521. The gate electrodes G1 and G2 may be formed above the channel regions of the semiconductor layers A1 and A2, that is, on positions of the gate insulating layer 521 overlapping the channel regions, respectively.

The interlayer insulating layer 522 may be disposed on the gate electrodes G1 and G2. The interlayer insulating layer 522 may include an inorganic insulating material such as silicon oxide (SiOₓ), silicon nitride (SiNₓ), silicon oxynitride (SiON), hafnium oxide (HfO₂), or aluminum oxide (Al₂O₃). In addition, the interlayer insulating layer 522 may include a plurality of insulating layers, and may further include a conductive layer disposed between the insulating layers and forming a capacitor second electrode.

The source electrodes S1 and S2 and the drain electrodes D1 and D2 are disposed on the interlayer insulating layer 522. A first source electrode S1 of the first thin film transistor TFT1 may be electrically connected to the source region of the first semiconductor layer A1 through a contact hole penetrating through the interlayer insulating layer 522 and the gate insulating layer 521. Similarly, a first drain electrode D1 of the first thin film transistor TFT1 may be electrically connected to the drain region of the first semiconductor layer A1 through a contact hole penetrating through the interlayer insulating layer 522 and the gate insulating layer 521. A second source electrode S2 of the second thin film transistor TFT2 may be electrically connected to the source region of the second semiconductor layer A2 through a contact hole penetrating through the interlayer insulating layer 522 and the gate insulating layer 521. Similarly, a second drain electrode D2 of the second thin film transistor TFT2 may be electrically connected to the drain region of the second semiconductor layer A2 through a contact hole penetrating through the interlayer insulating layer 522 and the gate insulating layer 521. Each of the source electrodes S1 and S2 and the drain electrodes D1 and D2 may include one or more metals selected from a group including aluminum (Al), molybdenum (Mo), platinum (Pt), palladium (Pd), silver (Ag), magnesium (Mg), gold (Au), nickel (Ni), neodymium (Nd), iridium (Ir), chromium (Cr), calcium (Ca), titanium (Ti), tantalum (Ta), tungsten (W), and copper (Cu).

A planarization layer 530 may be formed on the interlayer insulating layer 522 so as to cover each of the source electrodes S1 and S2 and the drain electrodes D1 and D2. The planarization layer 530 may be made of an organic insulating material or the like. The planarization layer 530 may have a flat top surface and include contact holes exposing any one of the source electrodes S1 and S2 and any one of the drain electrodes D1 and D2. A layer including components from the buffer layer 510 to the planarization layer 530 may be referred to as thin film transistor layer TFTL.

A light emitting element layer EML may be disposed on the planarization layer 530. The light emitting element layer EML may include light emitting elements EL, photoelectric conversion elements PD, and a bank layer BK. The light emitting element EL may include a pixel electrode 570, an emission layer 575, and a common electrode 590, and the photoelectric conversion element PD may include a first electrode 580, a photoelectric conversion layer 585, and a common electrode 590.

The pixel electrode 570 of the light emitting element EL may be disposed on the planarization layer 530. The pixel electrode 570 may be provided for each pixel PX. The pixel electrode 570 may be connected to the first source electrode S1 or the first drain electrode D1 of the first thin film transistor TFT1 through a contact hole penetrating through the planarization layer 530.

The pixel electrode 570 of the light emitting element EL may have a single-layer structure of, for example, molybdenum (Mo), titanium (Ti), copper (Cu), or aluminum (Al) or have a stacked film structure, for example, a multilayer structure of ITO/Mg, ITO/MgF₂, ITO/Ag, or ITO/Ag/ITO including indium-tin-oxide (ITO), indium-zinc-oxide (IZO), zinc oxide (ZnO), or indium oxide (In₂O₃), and silver (Ag), magnesium (Mg), magnesium fluoride (MgF₂), aluminum (Al), platinum (Pt), lead (Pb), gold (Au), or nickel (Ni), but the present disclosure is not limited thereto.

The first electrode 580 of the photoelectric conversion element PD may also be disposed on the planarization layer 530. The first electrode 580 may be provided for each photo-sensor PS. The first electrode 580 may be connected to the second source electrode S2 or the second drain electrode D2 of the second thin film transistor TFT2 through a contact hole penetrating through the planarization layer 530.

The first electrode 580 of the photoelectric conversion element PD may have a single-layer structure of, for example, molybdenum (Mo), titanium (Ti), copper (Cu), or aluminum (Al) or have a multilayer structure of, for example, ITO/Mg, ITO/MgF₂, ITO/Ag, or ITO/Ag/ITO, but the present disclosure is not limited thereto.

The bank layer BK may be disposed on the pixel electrode 570 and the first electrode 580. The bank layer BK may include openings formed in areas overlapping the pixel electrodes 570 and exposing the pixel electrodes 570. Areas in which the exposed pixel electrodes 570 and the emission layers 575 overlap each other may be defined as emission areas emitting different light according to the respective pixels PX: PX1, PX2, PX3, and PX4.

In addition, the bank layer BK may include openings formed in areas overlapping the first electrodes 580 and exposing the first electrodes 580. The openings exposing the first electrodes 580 may provide spaces in which the photoelectric conversion layers 585 of the respective photo-sensors PS are formed, and areas in which the exposed first electrodes 580 and the photoelectric conversion layers 585 overlap each other may be defined as light sensing parts.

The bank layer BK may include an organic insulating material such as, for example, a polyacrylates resin, an epoxy resin, a phenolic resin, a polyamides resin, a polyimides resin, an unsaturated polyesters resin, a polyphenyleneethers resin, a polyphenylenesulfides resin, or benzocyclobutene (BCB). As another example, the bank layer BK may also include an inorganic material such as, for example, silicon nitride (SiNₓ).

The emission layers 575 may be disposed on the pixel electrodes 570 of the light emitting elements EL exposed by the openings of the bank layer BK. The emission layer 575 may include a high molecular material or a low molecular material, and may emit red, green, or blue light for each pixel PX. The emission layer 575 may emit light in response to a potential difference between the pixel electrode 570 and the common electrode 590. For example, when an anode voltage is applied to the pixel electrode 570 and a cathode voltage is applied to the common electrode 590, the holes and electrons move to the emission layer 575, such that they combine in the emission layer 575 to emit light. The light emitted from the emission layer 575 may contribute to image display or function as a light source incident on the photo-sensor PS. For example, light sources of a green wavelength emitted from the emission areas of the second pixel PX2 and the fourth pixel PX4 may function as light sources incident on the light sensing areas of the photo-sensors PS.

When the emission layer 575 is made of an organic material, a hole injecting layer (HIL) and a hole transporting layer (HTL) may be disposed at a lower portion of each emission layer 575, and an electron injecting layer (EIL) and an electron transporting layer (ETL) may be stacked at an upper portion of each emission layer 575. Each of these layers may be a single layer or multiple layers made of an organic material.

The photoelectric conversion layers 585 may be disposed on the first electrodes 580 of the photoelectric conversion elements PD exposed by the openings of the bank layer BK. Areas in which the exposed first electrodes 580 and the photoelectric conversion layers 585 overlap each other may be defined as light sensing areas of the respective photo-sensors PS. The photoelectric conversion layer 585 may generate photocharges in proportion to incident light. The incident light may be light emitted from the emission layer 575 and then reflected to enter the photoelectric conversion layer 585 or may be light provided from the outside regardless of the emission layer 575. Charges generated and accumulated in the photoelectric conversion layer 585 may be converted into electrical signals required for sensing.

The photoelectric conversion layer 585 may include an electron donating material and an electron accepting material. The electron donating material may generate donor ions in response to light, and the electron accepting material may generate acceptor ions in response to light. When the photoelectric conversion layer 585 is made of an organic material, the electron donating material may include a compound such as, for example, subphthalocyanine (SubPc) or dibutylphosphate (DBP), but the present disclosure is not limited thereto. The electron accepting material may include a compound such as, for example, fullerene, a fullerene derivative, or perylene diimide, but the present disclosure is not limited thereto.

When the photoelectric conversion layer 585 is made of an inorganic material, the photoelectric conversion element PD may be a pn-type or pin-type phototransistor. For example, the photoelectric conversion layer 585 may have a structure in which an N-type semiconductor layer, an I-type semiconductor layer, and a P-type semiconductor layer are sequentially stacked. Here, the I-type semiconductor layer may include an intrinsic (pure) semiconductor which is a pure semiconductor without any significant dopant species present, and may also be called an undoped semiconductor or I-type semiconductor. The N-type semiconductor layer may include a semiconductor doped with N-type dopant. The N-type dopants may include, for example, phosphorus (P), arsenic (As), etc. The P-type semiconductor layer may include a semiconductor doped with P-type dopant. The P-type dopants may include, for example, boron (B), aluminum (Al), gallium (Ga), etc.

When the photoelectric conversion layer 585 is made of the organic material, a hole injecting layer (HIL) and a hole transporting layer (HTL) may be disposed at a lower portion of each photoelectric conversion layer 585, and an electron injecting layer (EIL) and an electron transporting layer (ETL) may be stacked at an upper portion of each photoelectric conversion layer 585. Each of these layers may be a single layer or multiple layers made of an organic material.

The common electrode 590 may be disposed on the emission layers 575, the photoelectric conversion layers 585, and the bank layer BK. The common electrode 590 may be disposed throughout the plurality of pixels PX and the plurality of photo-sensors PS in a form in which it covers the emission layers 575, the photoelectric conversion layers 585, and the bank layer BK. The common electrode 590 may include a conductive material having a small work function, for example, lithium (Li), calcium (Ca), lithium fluoride/calcium (LiF/Ca), lithium fluoride/aluminum (LiF/Al), aluminum (Al), magnesium (Mg), silver (Ag), platinum (Pt), palladium (Pd), nickel (Ni), gold (Au), neodymium (Nd), iridium (Ir), chromium (Cr), barium fluoride (BaF₂), barium (Ba), or compounds or mixtures thereof (e.g., a mixture of Ag and Mg, etc.). Alternatively, the common electrode 590 may include transparent metal oxide, for example, indium-tin-oxide (ITO), indium-zinc-oxide (IZO), or zinc oxide (ZnO).

The common electrode 590 may be disposed in common on the emission layer 575 and the photoelectric conversion layer 585, but the present disclosure is not limited thereto. In this case, a cathode electrode of the light emitting element EL and a sensing cathode electrode of the photoelectric conversion element PD may be electrically connected to each other. For example, a common voltage line connected to the cathode electrode of the light emitting element EL may also be connected to the sensing cathode electrode of the photoelectric conversion element PD.

An encapsulation layer TFEL may be disposed on the light emitting element layer EML. The encapsulation layer TFEL may include at least one inorganic layer to prevent oxygen or moisture from penetrating into each of the emission layer 575 and the photoelectric conversion layer 585. In addition, the encapsulation layer TFEL may include at least one organic layer to protect each of the emission layer 575 and the photoelectric conversion layer 585 from foreign materials such as dust. For example, the encapsulation layer TFEL may be formed in a structure in which a first inorganic layer 611, an organic layer 612, and a second inorganic layer 613 are sequentially stacked. Each of the first inorganic layer 611 and the second inorganic layer 613 may be formed as multiple layers in which one or more inorganic layers of, for example, a silicon nitride (SiNₓ) layer, a silicon oxynitride (SiON) layer, a silicon oxide (SiOₓ) layer, a titanium oxide (TiO₂) layer, and an aluminum oxide (Al₂O₃) layer are alternately stacked. The organic layer 612 may be an organic layer made of, for example, an acrylic resin, an epoxy resin, a phenolic resin, a polyamide resin, a polyimide resin, or the like. The encapsulation layer TFEL may seal the light emitting element layer EML. For example, the encapsulation layer TFEL may cover the light emitting element layer EML to prevent the light emitting element layer EML from being damaged or degraded by external impurities.

A pressure sensing layer PRS may be disposed on the encapsulation layer TFEL. The pressure sensing layer PRS may be provided in the form of a panel or a film, and may be attached onto the encapsulation layer TFEL through a bonding layer such as a pressure sensitive adhesive (PSA). The pressure sensing layer PRS is positioned on a light emission path of a display layer, and may thus be transparent.

The pressure sensing layer PRS serves to sense a pressure applied to the display device 1. When the user or the like touches an upper surface of the display device 1, a pressure applying force of a touch input may be sensed by the pressure sensing layer PRS. A pressure sensing electrode of the pressure sensing layer PRS may be directly formed on a touch layer. In this case, the pressure sensing layer PRS may be incorporated in the display panel 10 together with the display layer and the touch layer.

A window WDL may be disposed on the pressure sensing layer PRS. The window WDL may be disposed on the display device 1 to protect components of the display device 1, after a cutting process and a module process of the display cell 100 are performed. The window WM may be formed of a transparent material capable of outputting an image. The window WDL may be made of glass or plastic. An example in which the window WDL is implemented with a single layer is illustrated, but the present disclosure is not limited thereto. For example, the window WDL may include a plurality of layers. For example, the window WDL may have a laminated structure of a plurality of plastic films bonded with an adhesive, or may have a laminated structure of a glass substrate and a plastic film bonded with an adhesive.

FIG. 5 is a cross-sectional view illustrating a state in which a finger of a user is in contact with the window WDL of the display device 1, and when a finger or the like of a user OBJ is in contact with an upper surface of the window WDL, light output from the emission areas of the pixels PX may be reflected from the finger or the like of the user OBJ. In this case, blood flow rates according to a pressure in a blood vessel of the finger or the like of the user OBJ may be different from each other. Accordingly, the blood flow rate of the blood vessel of the finger or the like of the user OBJ may be derived based on a difference in an amount of the reflected light, that is, the light incident on the photo-sensors PS. A blood pressure of the user OBJ may be measured through the photo-sensors PS and the pressure sensing layer PRS.

FIG. 6 is a block diagram illustrating a main processor according to an embodiment of the present disclosure.

The main processor 800 includes the signal calculation unit 810, the blood pressure calculation unit 820, and the biometric authentication unit 830.

The signal calculation unit 810 includes a filter unit 811 and a feature point extraction unit 812.

The filter unit 811 may receive a pressure signal PSS having a pressure measurement value over time from the pressure sensing circuit 40. In addition, the filter unit 811 may receive a pulse wave signal PPG having a pulse wave measurement value over time from the pulse wave sensing circuit 50. The filter unit 811 may remove noise of the received pulse wave signal PPG. For example, the filter unit 811 may block a noise frequency of the pulse wave signal PPG using a low-pass filter, a high-pass filter, or a band-pass filter. The filter unit 811 may output the pulse wave signal PPG of which the noise is removed to the feature point extraction unit 812.

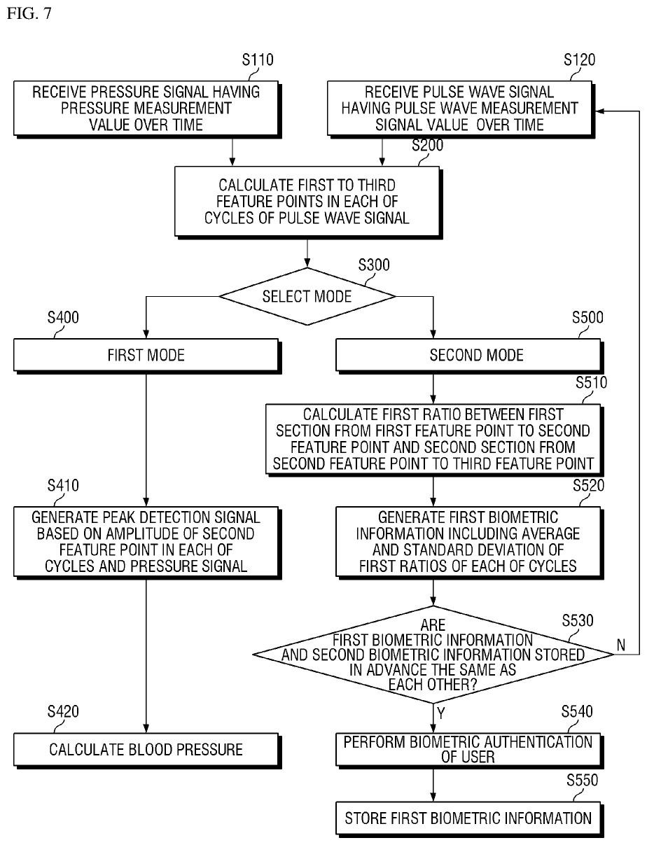

The feature point extraction unit 812 may receive the pulse wave signal PPG from the filter unit 811. The feature point extraction unit 812 may calculate feature points FF of the pulse wave signal PPG based on the received pulse wave signal PPG. For example, the feature point extraction unit 812 may calculate respective cycles of the pulse wave signal PPG and calculate amplitudes of the respective cycles. In addition, the feature point extraction unit 812 may calculate peak points of the pulse wave signal PPG reflecting a blood change depending on a heartbeat in the respective cycles of the pulse wave signal PPG as the feature points FF. The feature point extraction unit 812 may output the calculated feature points FF to the blood pressure calculation unit 820 and the biometric authentication unit 830. A detailed description of the feature point FF will be described later with reference to FIGS. 7 to 12.

The blood pressure calculation unit 820 includes a peak detection unit 821 and a blood pressure information generation unit 822. The blood pressure calculation unit 820 may calculate blood pressure information of the user in the first mode.

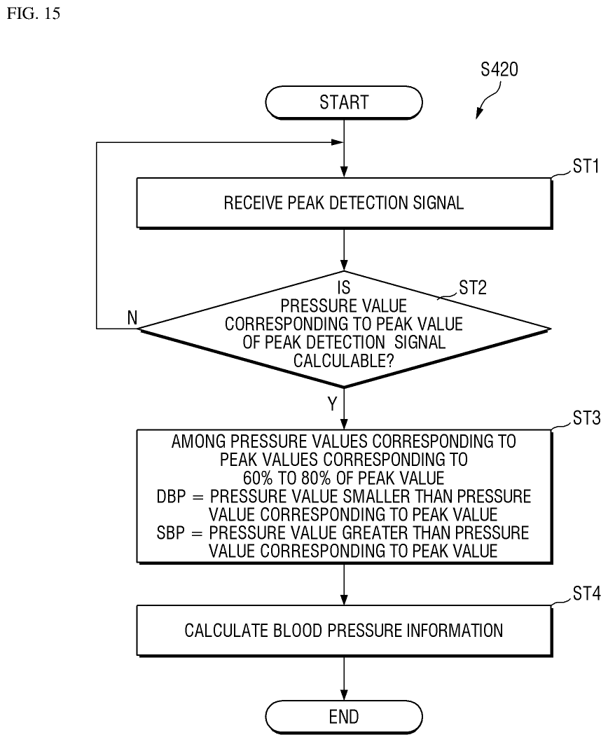

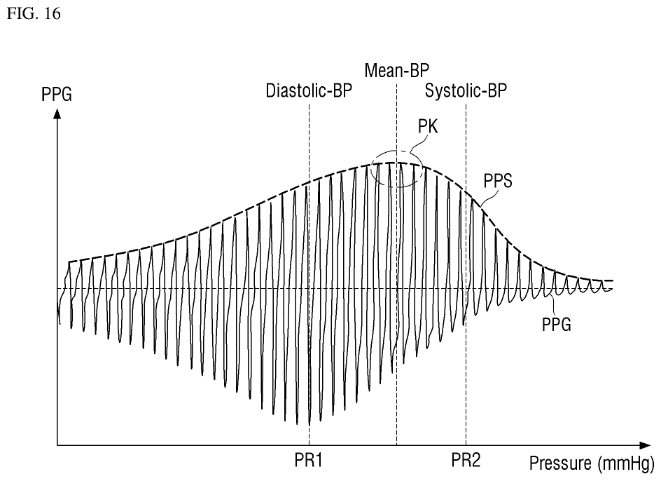

The peak detection unit 821 may receive data of the feature point FF from the signal calculation unit 810 in the first mode. The peak detection unit 821 may generate a peak detection signal PPS based on the received data of the feature point FF. For example, each of cycles of the pulse wave signal PPG may include a plurality of waveforms, and a pulse wave signal PPG value of a first waveform of the plurality of waveforms may be calculated as an amplitude. In addition, information on the respective cycles of the pulse wave signal PPG may also be calculated as the feature point FF. Accordingly, the data of the feature point FF received by the peak detection unit 821 may include data on a cycle and an amplitude of the pulse wave signal PPG. The peak detection unit 821 may generate the peak detection signal PPS based on the data on the cycle and the amplitude of the pulse wave signal PPG. The peak detection unit 821 may output the generated peak detection signal PPS to the blood pressure information generation unit 822. In addition, the blood pressure information generation unit 822 may calculate a blood pressure based on a peak value of the received peak detection signal PPS. This will be described later with reference to FIGS. 15 and 16.

The biometric authentication unit 830 includes a biometric information generation unit 831 and a biometric information authentication unit 832. The biometric authentication unit 830 may generate biometric information for each user and compare the generated biometric information with biometric information stored in the memory 900 to perform biometric authentication, in the second mode.

The biometric information generation unit 831 may receive data of the feature point FF from the signal calculation unit 810 in the second mode. The biometric information generation unit 831 may generate first biometric information BS1 based on the received data of the feature point FF. For example, each of cycles of the pulse wave signal PPG may include a plurality of waveforms, and a pulse wave signal PPG value of a first waveform of the plurality of waveforms may be calculated as an amplitude. Alternatively, an inflection point of a waveform formed within a cycle of the pulse wave signal PPG may be calculated as the feature point FF. Accordingly, the data of the feature point FF received by the biometric information generation unit 831 may include biometric information of the user formed within the cycle of the pulse wave signal PPG. The biometric information generation unit 831 may generate the first biometric information BS1 based on the data of the feature point FF. The biometric information generation unit 831 may output the generated first biometric information BS1 to the biometric information authentication unit 832.

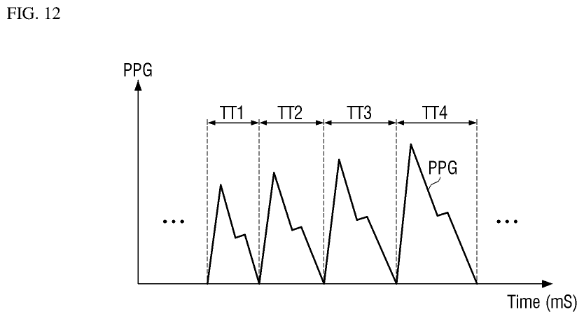

The biometric information authentication unit 832 may receive the first biometric information BS1 from the biometric information generation unit 831. In addition, the biometric information authentication unit 832 may receive second biometric information BS2 of a user stored in advance in the memory 900 from the memory 900. The biometric information authentication unit 832 may decide whether or not the first biometric information BS1 and the second biometric information BS2 are the same as each other. For example, when the first biometric information BS1 and the second biometric information BS2 are the same as each other, the biometric information authentication unit 832 may decide that the user of the second biometric information BS2 stored in advance in the memory 900 and the user of the first biometric information BS1 are the same as each other. Alternatively, when the first biometric information BS1 and the second biometric information BS2 are not the same as each other, the biometric information authentication unit 832 may not perform biometric authentication. This will be described later with reference to FIGS. 7 and 12.

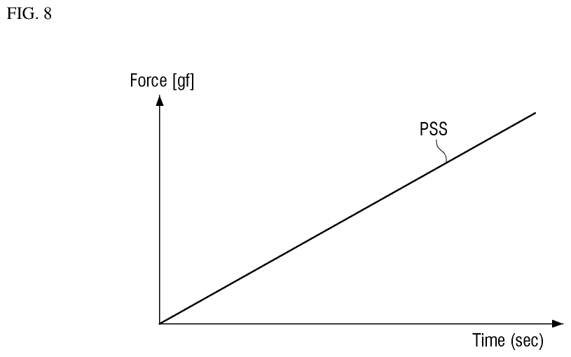

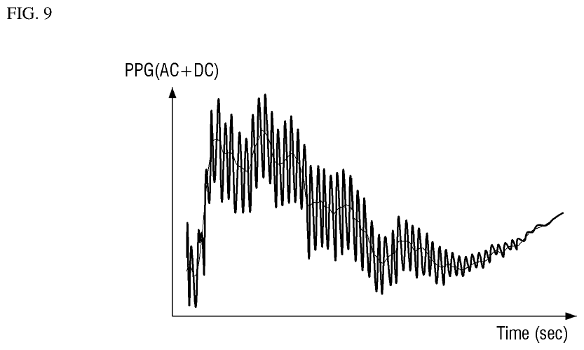

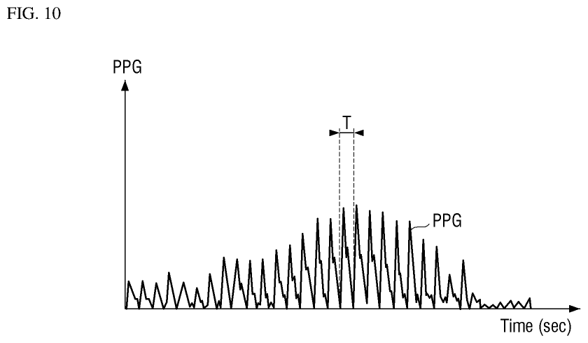

FIG. 7 is a flowchart illustrating a blood pressure measurement and biometric authentication method according to an embodiment of the present disclosure. FIG. 8 is a graph of a pressure signal indicating a pressure measurement value over time. FIGS. 9 and 10 are graphs of a pulse wave signal indicating a pulse wave measurement value over time. FIG. 11 is a graph illustrating one cycle of the pulse wave signal of FIG. 10. FIG. 12 is a graph illustrating cycles of a pulse wave signal.

Referring to FIG. 7, first, the main processor 800 may receive a pressure signal PSS sensed by the pressure sensor, generated by the pressure sensing circuit 40 and having a pressure measurement value over time (S110).

Referring further to FIG. 8, the user may apply a pressure to a position where the pressure sensor is disposed, and the pressure sensor may measure a pressure measurement value of the pressure applied by the user. For example, the graph in FIG. 8 may be plotted with the change in force (with gram-force (gf) unit) verses the change in time (with second (sec) unit). In an embodiment of the present disclosure, the pressure sensor may be located in the pressure sensing layer PRS (see FIG. 5) of a display cell 100 (see FIG. 4). A method of generating the pulse wave signal PPG will be described in detail. For example, in a process in which the user brings his or her finger into contact with the display device 1, the pressure measurement value measured by the pressure sensor may gradually increase over time to reach a maximum value. When the pressure measurement value (i.e., a contact pressure) increases, a blood vessel may be constricted, such that a blood flow rate may be decreased or become 0. Accordingly, the main processor 800 may receive the pressure signal PSS having the pressure measurement value over time, generated by the pressure sensing circuit 40.

The main processor 800 may receive a pulse wave signal PPG having a pulse wave measurement value over time (S120).

Referring further to FIGS. 9 and 10, to generate the pulse wave signal PPG, pulse wave information over time is also required together with the pressure data. During systole of the heart, blood ejected from the left ventricle of the heart moves to peripheral tissues, such that a blood volume in the arterial side increases. In addition, during the systole of the heart, red blood cells carry more oxyhemoglobin to the peripheral tissues. During diastole of the heart, there is partial suction of blood from the peripheral tissues toward the heart. In this case, when a peripheral blood vessel is irradiated with light emitted from a display pixel, the irradiated light may be absorbed by the peripheral tissue. Absorbance is dependent on a hematocrit and a blood volume. The absorbance may have a maximum value during the systole of the heart and a minimum value during the diastole of the heart. Since the absorbance is in inverse proportion to an amount of light incident on the photo-sensor PS, absorbance at a corresponding point in time may be estimated through light reception data of the amount of light incident on the photo-sensor PS, and accordingly, as illustrated in FIG. 9, a pulse wave signal PPG value over time may be generated.

The pulse wave information over time reflects the maximum value of the absorbance during the systole of the heart, and reflects the minimum value of the absorbance during the diastole of the heart. In addition, the pulse wave vibrates according to a heartbeat cycle T. Accordingly, the pulse wave information may reflect a change in blood pressure depending on a heartbeat. Accordingly, the pulse wave sensing circuit 50 may measure the pulse wave signal PPG value according to a pressure applying time. However, the pulse wave signal PPG may include both an alternating current (AC) component and a direct current (DC) component. The filter unit may remove the DC component from the pulse wave signal PPG to generate a pulse wave signal PPG (see FIG. 10) plotted according to a magnitude of a time. Accordingly, the pulse wave signal PPG represents a pulse wave AC component according to a pressure.

Next, the main processor 800 calculates first to third feature points F1 to F3 in each of cycles T of the pulse wave signal PPG (S200).

The main processor 800 may calculate feature points FF in each of the cycles T of the pulse wave signal PPG to calculate a blood pressure in the first mode and perform biometric authentication for each user in the second mode. The feature points FF refer to points of the pulse wave signal PPG at which blood pressure information for each user may be calculated and biometric authentication for each user may be performed. In this case, a gender, an age, and a health state may be different for each user, and accordingly, states of the hearts, blood vessels, and autonomic nerves may also be different for each user. As described above, the pulse wave signal PPG includes biometric information of a hematocrit of a blood vessel and of a blood volume according to a heart rate of the heart, and thus, a waveform of the pulse wave signal PPG and feature points FF according to the waveform may be different for each user. In addition, the pulse wave signal PPG per unit cycle T may have signal waveforms having a substantially similar shape. Accordingly, the feature points FF may be calculated to compare the waveforms of the pulse wave signal PPG per unit cycle T with each other and apply a correlation according to a difference between the waveforms of the pulse wave signal PPG per unit cycle T.

Hereinafter, a method of calculating the feature point FF of the pulse wave signal PPG in an embodiment of the present disclosure will be described.

Referring further to FIG. 11, the pulse wave signal PPG may have respective cycles T and feature points FF. One cycle T of the pulse wave signal PPG may be defined as, for example, a time from the lowest point to the next lowest point. In addition, when a waveform of the pulse wave signal PPG is viewed in units of the cycle T, signal waveforms having a substantially similar shape may be repeated in the pulse wave signal PPG.

The feature points FF may be defined by inflection points of a waveform formed within one cycle T. For example, the feature points FF may include a first feature point F1 positioned at the lowest point and a third feature point F3 positioned at the lowest point next to the lowest point which the first feature point F1 positioned at, in one cycle T of the pulse wave signal PPG. The lowest point of the first feature point F1 and the lowest point of the third feature point F3 may be located at about the same level (see FIGS. 10-12). In this case, one cycle T may be defined as a time from the first feature point F1 to the third feature point F3. In addition, the feature points FF may include a second feature point F2 positioned at the highest point in one cycle T of the pulse wave signal PPG. The second feature point F2 may be an upward convex peak in one cycle T of the pulse wave signal PPG. At the second feature point F2, the pulse wave signal PPG may have an amplitude V1 in one cycle T of the pulse wave signal PPG. The amplitude V1 may be a magnitude of the pulse wave signal PPG in one cycle T. In this case, the amplitude V1 may be a difference between a value of the pulse wave signal PPG at the first feature point F1 and a value of the pulse wave signal PPG at the second feature point F2.

The present disclosure is not limited to that described above, and the feature points FF may further include points having blood pressure information and biometric information for each user. For example, the feature points FF may further include a fourth feature point F4 positioned between the second feature point F2 and the third feature point F3 in a second section T2 and downward convex and a fifth feature point F5 positioned between the second feature point F2 and the third feature point F3 in the second section T2 and upward convex.

In summary, the main processor 800 may calculate the feature points FF to calculate the blood pressure for each user based on the waveform of the pulse wave signal PPG per unit cycle T or to perform the biometric authentication for each user based on the waveform of the pulse wave signal PPG per unit cycle T.

Referring to FIG. 7 again, next, a mode may be selected (S300).

When the first mode is selected (S400), a peak detection signal PPS (see FIG. 16) is generated based on the amplitude V1 of the second feature point F2 in each of the cycles T and the pressure signal PSS (S410). In addition, the main processor 800 may calculate the blood pressure based on the peak detection signal PPS (see FIG. 16) (S420).

The main processor 800 may receive the feature points FF and the amplitude V1 calculated in each of the cycles T of the pulse wave signal PPG. In addition, the main processor 800 may receive the pressure signal PSS having the pressure measurement value over time from the pressure sensing circuit 40. The main processor 800 may generate the peak detection signal PPS (see FIG. 16) having a magnitude of the pulse wave signal PPG according to a pressure based on the received feature points FF, amplitude V1, and pressure signal PSS. For example, the peak detection signal PPS (see FIG. 16) is defined as a signal corresponding to the amplitude V1 of each of the cycles of the pulse wave signal PPG. That is, the peak detection signal PPS (see FIG. 16) may be defined as a signal corresponding to a peak value of each of the cycles of the pulse wave signal PPG. For example, the pulse wave signal PPG may have one or more amplitudes V1. The main processor 800 may calculate the peak detection signal PPS (see FIG. 16) including points corresponding to the amplitude V1 of the respective cycles T of the pulse wave signal PPG. That is, the generated peak detection signal PPS (see FIG. 16) may be a signal having the amplitude V1 according to a pressure. A method of calculating the blood pressure based on the peak detection signal PPS (see FIG. 16) will be described later with reference to FIGS. 15 and 16.

A biometric authentication method of the user when the second mode is selected (S500) will hereinafter be described in detail.

First, the main processor 800 calculates a first ratio between a first section T1 from the first feature point F1 to the second feature point F2 and a second section T2 from the second feature point F2 to the third feature point F3 in each of the cycles T of the pulse wave signal PPG (S510).

Referring further to FIG. 11, each cycle T of the pulse wave signal PPG may include the first section T1 (or a rising section) from the first feature point F1 to the second feature point F2 and the second section T2 (or a falling section) from the second feature point F2 to the third feature point F3. The main processor 800 may calculate the first section T1 and the second section T2 based on the first feature point F1 to the third feature point F3 calculated in each of the cycles T of the pulse wave signal PPG and calculate the first ratio between the first section T1 and the second section T2. For example, the first ratio may be a ratio of a time of the second section T2 to a time of the first section T1. The first ratio between the first section T1 and the second section T2 may have a correlation with a time ratio between the diastole and the systole of the heart in one cycle T of the pulse wave signal PPG. In addition, the first ratio may have a different value according to a heart, a blood vessel, and an autonomic nerve for each user. That is, since the first ratio between the first section T1 and the second section T2 is different for each user, the first ratio may be used as information for performing the biometric authentication for each user.

The main processor 800 may calculate another ratio based on the feature points FF. For example, the main processor 800 may calculate a ratio between a first area AR1 and a second area AR2 as information for performing the biometric authentication for each user. For example, an area AR of one cycle T may include the first area AR1 of the first section T1 and the second area AR2 of the second section T2. In this case, the area AR may be calculated as an area between the waveform and the line connecting the lowest point. The main processor 800 may calculate the first area AR1 and the second area AR2 based on the calculated first and third feature points F1 to F3 and calculate a second ratio between the first area AR1 and the second area AR2. Since the second ratio is also different according to the heart, the blood vessel, and the autonomic nerve for each user, the second ratio may be used as information for performing the biometric authentication for each user.

In an embodiment of the present disclosure, a method of calculating the first ratio based on the first feature point F1 to the third feature point F3 has been described by way of example, but the present disclosure is not limited thereto, and the main processor 800 may also calculate a ratio including biometric authentication information for each user based on the fourth feature point F4 and the fifth feature point F5. For example, the main processor 800 may also calculate a ratio between a section from the second feature point F2 to the fourth feature point F4, a section from the fourth feature point F4 to the fifth feature point F5, and a section from the fifth feature point F5 to the third feature point F3 and the first section T1 or the second section T2 as the first ratio.

Second, the main processor 800 generates first biometric information BS1 including the first ratios of each of the cycles T of the pulse wave signal PPG, an average of the first ratios, and a standard deviation of the first ratios (S520).

Referring further to FIG. 12, the main processor 800 may generate the first biometric information BS1 based on the first ratios of each of the cycles T of the pulse wave signal PPG. For example, when the respective cycles T of the pulse wave signal PPG have different times, the respective cycles T of the pulse wave signal PPG may have different widths. In this case, the respective cycles T of the pulse wave signal PPG are changed depending on a heart rate of the user. For example, since the heart rate of the user may be changed instantaneously depending on exercise, breathing, a mental state, and the like, the respective cycles T of the pulse wave signal PPG may have different widths. On the other hand, even though the respective cycles T of the pulse wave signal PPG have different widths, the first ratios of the respective cycles T of the pulse wave signal PPG may be irrelevant to a width of the cycle T. For example, referring to a case of the pulse wave signal PPG having different cycles T in FIG. 12, a width of the cycle T sequentially increases from a first cycle TT1 to a fourth cycle TT4 of the pulse wave signal PPG. In this case, first ratios of the first cycle TT1 to the fourth cycle TT4 may not be affected by the width of the cycle T. Accordingly, the main processor 800 may generate the first biometric information BS1 for performing the biometric authentication for each user based on the first ratios of each of the cycles T of the pulse wave signal PPG.

The main processor 800 may generate the first biometric information BS1 by calculating the average and the standard deviation of the first ratios of each of the cycles T of the pulse wave signal PPG. For example, the first ratio calculated in each cycle T of the pulse wave signal PPG may have different values for each cycle T. According to health, a blood vessel, an autonomic nerve, and the like, of the user, the first ratio for each cycle T of the pulse wave signal PPG may have a predetermined tendency or may have a cyclic property. Accordingly, the average and the standard deviation of the first ratios of each of the cycles T of the pulse wave signal PPG may be different for each user. Accordingly, the main processor 800 may calculate the average and the standard deviation of the first ratios of each of the cycles T of the pulse wave signal PPG and generate the first biometric information BS1 for performing the biometric authentication for each user based on the average and the standard deviation of the first ratios. Alternatively, the main processor 800 may calculate a second ratio between an area of the pulse wave signal PPG in the first section T1 and an area of the pulse wave signal PPG in the second section T2 in the second mode, and the first biometric information BS1 may include information on an average and a standard deviation of the second ratios for performing the biometric authentication for each user.

In summary, in an embodiment of the present disclosure, the first ratios calculated per unit cycle T include information for performing the biometric authentication for each user. Accordingly, the main processor 800 may generate the first biometric information BS1 for performing the biometric authentication for each user based on the first ratios calculated per unit cycle T.

Third, the main processor 800 may decide whether or not the first biometric information BS1 and second biometric information BS2 stored in advance in the memory 900 are the same as each other (S530), and perform the biometric authentication of the user (S540).

The first biometric information BS1 generated by the main processor 800 includes the first ratios calculated per unit cycle T of the pulse wave signal PPG, the average of the first ratios, and the standard deviation of the first ratios. In addition, the second biometric information BS2 stored in advance in the memory 900 includes first ratios calculated per unit cycle T of the pulse wave signal PPG of the user, an average of the first ratios, and a standard deviation of the first ratios. The main processor 800 may decide whether or not the first ratios, the average of the first ratios, and the standard deviation of the first ratios of the first biometric information BS1 are the same as the first ratios, the average of the first ratios, and the standard deviation of the first ratios of the second biometric information BS2, respectively. When the first biometric information BS1 and the second biometric information BS2 are the same as each other, the main processor 800 may recognize the user having the first biometric information BS1 as the same user as a user having the second biometric information BS2. Accordingly, the main processor 800 may execute a designated application or the like according to the biometric authentication of the user. On the other hand, when the first biometric information BS1 and the second biometric information BS2 are not the same as each other, the main processor 800 may recognize the user having the first biometric information BS1 as a user different from the user having the second biometric information BS2.

After performing the blood pressure measurement and biometric authentication, the blood pressure information may be displayed on a display panel 10 in the first mode, and a decision result for whether or not the first biometric information BS1 coincides with the second biometric information BS2 may be displayed on the display panel 10 in the second mode.

Meanwhile, the main processor 800 may store the first biometric information BS1 in the memory 900 (S550).

When the first biometric information BS1 is the same as the second biometric information BS2, the main processor 800 may store the first biometric information BS1 in the memory 900. For example, the main processor 800 may be configured to store the first biometric information BS1 in the memory 900 when the first biometric information BS1 coincides with the second biometric information BS2 stored in the memory 900. The main processor 800 decides that the first biometric information BS1 is biometric information of an authenticated user. Accordingly, when the biometric information of the user is authenticated, the main processor 800 may store the first biometric information BS1 in the memory 900 to use the first biometric information B Si as the biometric information of the user. Accordingly, the memory 900 may store the received first biometric information BS1 as new second biometric information BS2.

In the present embodiment, the main processor 800 may receive the pulse wave signal PPG and calculate the feature point FF. In addition, the main processor 800 may calculate the blood pressure based on the calculated feature point FF and the received pressure signal PSS in the first mode, and may calculate information capable of performing the biometric authentication for each user and perform the biometric authentication in the second mode. That is, the display device 1 may calculate the blood pressure information in the first mode and perform the biometric authentication for each user without a separate biometric authentication module in the second mode by calculating the feature points FF for each user, of the pulse wave signal PPG sensed by the photo-sensor.

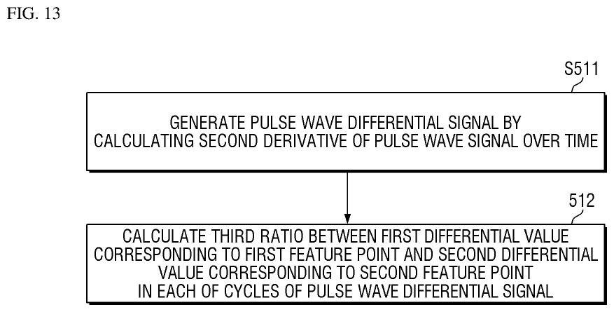

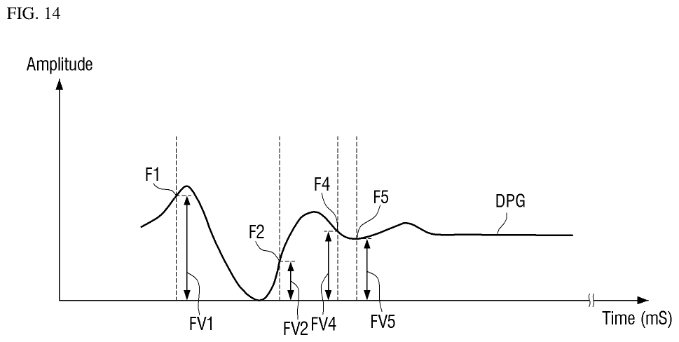

FIG. 13 is a flowchart illustrating a method of calculating a third ratio according to an embodiment of the present disclosure. FIG. 14 is a graph illustrating a waveform of a pulse wave differential function.

The embodiment of FIGS. 13 and 14 is different from the embodiment of FIGS. 7 to 12 in that a pulse wave differential signal DPG is generated by calculating a second derivative of the pulse wave signal PPG over time and the first biometric information BS1 is generated by calculating a third ratio according to the pulse wave differential signal DPG. A method of calculating the third ratio by the main processor 800 in the embodiment of FIGS. 13 and 14 will be described.

Referring to FIG. 13, the main processor 800 may calculate a second derivative of the pulse wave signal PPG (S511).

Referring further to FIG. 14, for example, the main processor 800 may calculate the second derivative of the pulse wave signal PPG over time and plot a graph of the pulse wave differential signal DPG. In this case, the pulse wave signal PPG may be referred to as a first pulse wave signal, and the pulse wave differential signal DPG may be referred to as a second pulse wave signal. As illustrated in FIG. 14, the pulse wave differential signal DPG may have a plurality of inflection points. Likewise, the main processor 800 may derive a graph having a plurality of inflection points of the pulse wave differential signal DPG. The pulse wave differential signal DPG is a signal reflecting an acceleration of the pulse wave signal PPG, and may be a second derivative of photoplethysmography (SDPTG) signal. The plurality of inflection points of the pulse wave differential signal DPG may include body information such as a degree of elasticity of a blood vessel, an aging degree of the blood vessel, and arteriosclerosis of the user.

Next, the main processor 800 may calculate a third ratio between a first differential value FV1 corresponding to a first feature point F1 and a second differential value FV2 corresponding to a second feature point F2 in one cycle of the pulse wave differential signal DPG (S512).

As described above, the pulse wave signal PPG may have a different value according to the heart, the blood vessel, and the autonomic nerve for each user, and the pulse wave differential signal DPG may also have a different value for each user. In addition, a value of the pulse wave differential signal DPG represents acceleration information of the pulse wave signal PPG, and the pulse wave differential signal DPG may have a different value according to body information such as a degree of elasticity of a blood vessel, an aging degree of the blood vessel, and arteriosclerosis for each user. Accordingly, in the pulse wave differential signal DPG, the first differential value FV1 of the first feature point F1 and the second differential value FV2 of the second feature point F2 may be different for each user. Accordingly, the main processor 800 may calculate the first differential value FV1 and the second differential value FV2 and calculate the third ratio between the first differential value FV1 and the second differential value FV2. Since the third ratio is also different for each user, the third ratio may be used as information for performing biometric authentication for each user. However, the present disclosure is not limited thereto, and the main processor 800 may further include information on a fourth differential value FV4 of a fourth feature point F4 and a fifth differential value FV5 of a fifth feature point F5 to calculate the third ratio.

The present embodiment may be helpful in increasing accuracy of the biometric authentication through the third ratio of the pulse wave differential signal DPG. For example, when the main processor 800 generates the first biometric information BS1 as in the embodiment of FIGS. 7 to 12, the first biometric information BS1 may further include the third ratio according to the present embodiment in addition to the first ratio. Accordingly, an error occurrence probability in the first biometric information BS1 may be reduced.

FIG. 15 is a flowchart illustrating a method of calculating a blood pressure according to an embodiment of the present disclosure. FIG. 16 is a graph illustrating a waveform of a peak detection signal. A method of calculating a blood pressure based on the feature point FF will be described with reference to FIGS. 15 and 16.

Referring further to FIGS. 15 and 16, first, the main processor 800 receives a peak detection signal PPS (ST1). Next, the main processor 800 decides whether or not a pressure value corresponding to a peak value PK of the peak detection signal PPS may be calculated (ST2). When a peak of the peak detection signal PPS exists, the main processor 800 may calculate the pressure value corresponding to the peak value PK of the peak detection signal PPS. The pressure value corresponding to the peak value PK of the peak detection signal PPS may be referred to as PK pressure value.

Next, the main processor 800 calculates a systolic blood pressure SBP, a diastolic blood pressure DBP, and the like, based on the peak value PK of the peak detection signal PPS (ST3), and calculates blood pressure information (ST4).

The main processor 800 may calculate the diastolic blood pressure DBP lower than the PK pressure value, the systolic blood pressure SBP higher than the PK pressure value, and a mean blood pressure (Mean-BP in FIG. 16) according to the PK pressure value. For example, the main processor 800 may calculate pressure values corresponding to values of the peak detection signal PPS corresponding to 60% to 80% of the peak value PK. The main processor 800 may calculate a pressure value smaller than the PK pressure value among the pressure values, which correspond to values of the peak detection signal PPS corresponding to 60% to 80% of the peak value PK, as a first pressure value PR1. In addition, the main processor 800 may calculate the first pressure value PR1 as the diastolic blood pressure DBP (Diastolic-BP in FIG. 16). In addition, the main processor 800 may calculate a pressure value greater than the PK pressure value among the pressure values, which correspond to values of the peak detection signal PPS corresponding to 60% to 80% of the peak value PK, as a second pressure value PR2. In addition, the main processor 800 may calculate the second pressure value PR2 as the systolic blood pressure SBP (Systolic-BP in FIG. 16).

In a case of the present embodiment, the pulse wave signal PPG vibrates according to the heartbeat cycle, and may thus reflect a change in blood pressure according to the heartbeat. The display device 1 may accurately calculate the blood pressure information based on the second feature point F2 and the amplitude V1 of the pulse wave signal PPG.

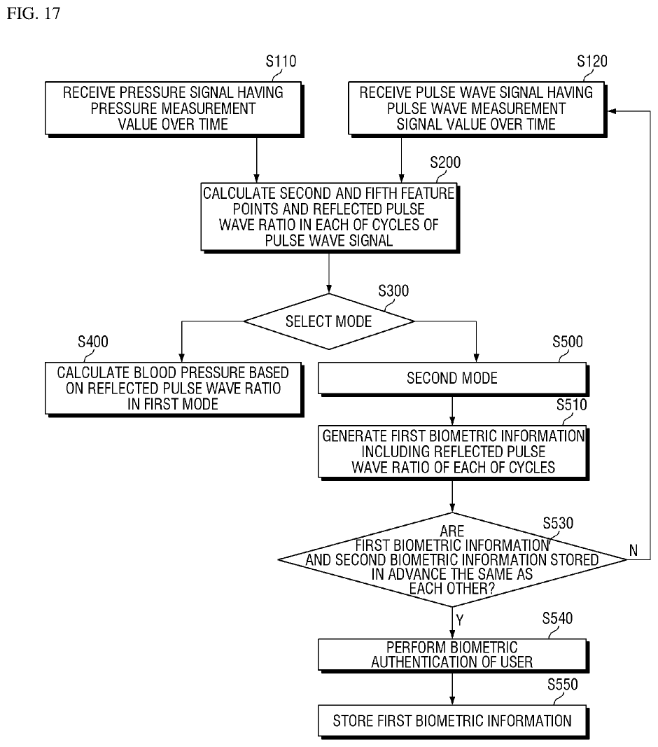

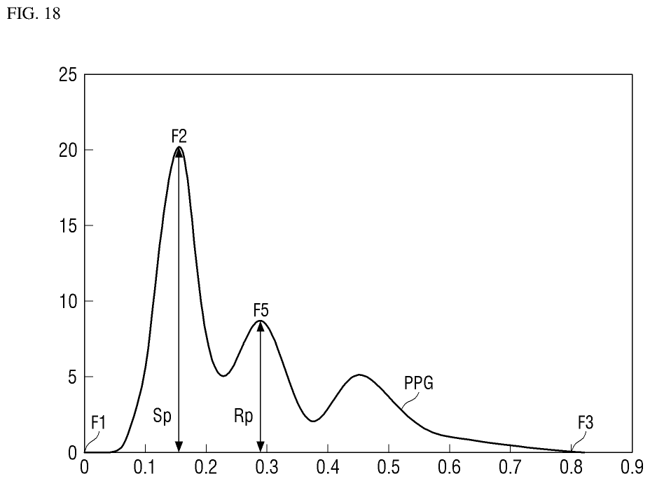

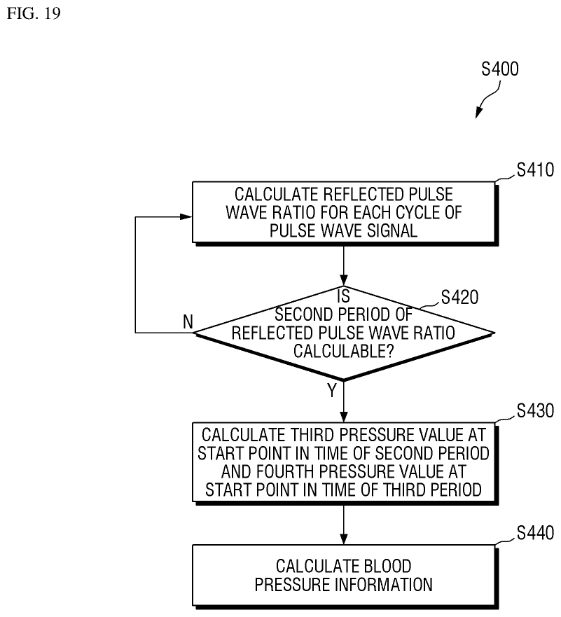

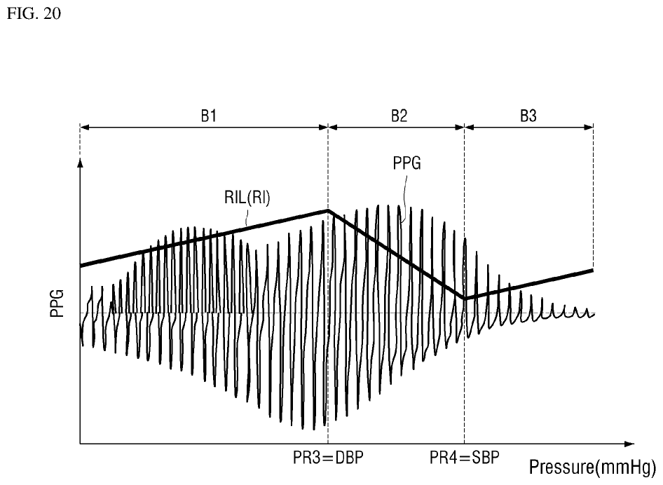

FIG. 17 is a flowchart illustrating a blood pressure measurement and biometric authentication method according to an embodiment of the present disclosure. FIG. 18 is a graph illustrating a waveform of one cycle of a pulse wave signal. FIG. 19 is a flowchart illustrating a method of calculating a blood pressure according to an embodiment of the present disclosure. FIG. 20 is a graph illustrating a pulse wave signal and a reflected pulse wave ratio according to an embodiment of the present disclosure.

The embodiment of FIGS. 17 to 20 is different from the embodiment of FIGS. 7 to 12 in that a blood pressure may be calculated in the first mode and biometric authentication may be performed in the second mode, using a reflected pulse wave ratio. Hereinafter, contents different from those of the embodiment of FIGS. 7 to 12 will be mainly described.

Referring to FIG. 17, the main processor 800 may receive a pressure signal PSS sensed by the pressure sensor, generated by the pressure sensing circuit 40 and having a pressure measurement value over time (S110). In addition, the main processor 800 may receive a pulse wave signal PPG having a pulse wave measurement value over time (S120). A description thereof is substantially the same as that of the embodiment of FIGS. 7 to 12, and will thus be omitted.

Next, the main processor 800 calculates a reflected pulse wave ratio RI in each of cycles T of the pulse wave signal PPG (S200).

Referring to FIG. 18, the main processor 800 may calculate the reflected pulse wave ratio RI of the pulse wave signal PPG. To calculate the reflected pulse wave ratio RI, the main processor 800 divides a wave cycle of the pulse wave signal PPG according to a period in which a wave according to a heartbeat and a reflected wave of a blood vessel are sequentially generated. For example, one cycle of the pulse wave signal PPG may include a plurality of waveforms having different amplitudes. Accordingly, when a peak value of a waveform having the greatest amplitude among the plurality of waveforms is defined as a pulse wave contraction value, a peak value of a waveform having the second greatest amplitude among the plurality of waveforms is defined as a reflected pulse wave value, the pulse wave contraction value is defined as Sp, the reflected pulse wave value is defined as Rp, and the reflected pulse wave ratio is defined as RI, the reflected pulse wave ratio RI may be calculated by the following Equation 1.

$$R I = \frac{R p}{S p} \qquad Equation 1$$

Here, the pulse wave contraction value Sp may have the same value as an amplitude V1 of a second feature point F2. That is, the pulse wave contraction value Sp may be a difference between a value of the first feature point F1 and a value of the second feature point F2. In addition, the peak value of the waveform having the greatest amplitude may be substantially the same as the second feature point F2, and the peak value Rp of the waveform having the second greatest amplitude may be substantially the same as a fifth feature point F5.

In summary, the main processor 800 may calculate the second feature point F2 corresponding to the peak value Sp of the waveform having the greatest amplitude among the plurality of waveforms of one cycle of the pulse wave signal PPG. In addition, the main processor 800 may calculate the fifth feature point F5 corresponding to the peak value Rp of the waveform having the second greatest amplitude among the plurality of waveforms of one cycle of the pulse wave signal PPG. In addition, the main processor 800 may calculate the reflected pulse wave ratio RI based on the second feature point F2 and the fifth feature point F5.

Next, a mode may be selected (S300), and the main processor 800 may calculate a blood pressure based on the reflected pulse wave ratio RI in the first mode (S400).

Referring further to FIGS. 19 and 20, first, a reflected pulse wave ratio RI is calculated for each cycle of the pulse wave signal PPG (S410). Second, the main processor 800 decides whether or not a second period B2 of the reflected pulse wave ratio RI may be calculated (S420). The main processor 800 sequentially stores detection results of reflected pulse wave ratios RI of reflected pulse waves to pulse wave contraction values, and analyzes the stored reflected pulse wave ratios RI. The main processor 800 may continuously convert changes in magnitude of the reflected pulse wave ratios RI into data to analyze a change in magnitude of reflected pulse wave ratio data RIL(RI).

The reflected pulse wave ratio RI includes a first period B1 in which the reflected pulse wave ratio RI fluctuates within a first range, a second period B2 in which the reflected pulse wave ratio RI fluctuates within a second range, and a third period B3 in which the reflected pulse wave ratio RI fluctuates within a third range. For example, the main processor 800 may analyze a reflected pulse wave ratio signal RIL to analyze a first period B1 in which the reflected pulse wave ratio RI is gently changed within a preset range in a saturated state, a second period B2 in which the reflected pulse wave ratio RI is sharply decreased or increased beyond the preset range within a preset period, a third period B3 in which the reflected pulse wave ratio RI is gently changed within the preset range in a saturated state again after it is sharply decreased or increased, and the like.

Here, a width of the first range and a width of the third range may be smaller than a width of the second range. In addition, a gradient of the second period B2 of the reflected pulse wave ratio RI may be greater than a gradient of the first period B1 of the reflected pulse wave ratio RI and a gradient of the third period B3 of the reflected pulse wave ratio RI.

Finally, the main processor 800 calculates a systolic blood pressure SBP, a diastolic blood pressure DBP, and the like, based on the reflected pulse wave ratio RI (S430), and calculates blood pressure information (S440).

The main processor 800 may analyze the reflected pulse wave ratio RI to detect a start point in time of the second period B2. In addition, the main processor 800 may calculate a third pressure value PR3 corresponding to the pulse wave signal PPG at the start point in time of the second period B2. In addition, the main processor 800 may calculate the third pressure value PR3 as the diastolic blood pressure DBP. In addition, the main processor 800 may analyze the reflected pulse wave ratio RI to detect a start point in time of the third period B3 after the second period B2. In addition, the main processor 800 may calculate a fourth pressure value PR4 corresponding to the pulse wave signal PPG at the start point in time of the third period B3. In addition, the main processor 800 may calculate the fourth pressure value PR4 as the systolic blood pressure SBP.

Referring to FIG. 17 again, the main processor 800 may generate the first biometric information BS1 based on the reflected pulse wave ratio RI (S510). For example, the reflected pulse wave ratio RI may have a correlation with a ratio between the diastole and the systole of the heart, the degree of elasticity of the blood vessel, whether or not arteriosclerosis exists, and the like, in one cycle T of the pulse wave signal PPG. That is, the reflected pulse wave ratio RI may be different for each user. Accordingly, the reflected pulse wave ratio RI may also be used as information for performing the biometric authentication for each user.

Third, the main processor 800 may decide whether or not the first biometric information BS1 and second biometric information BS2 stored in advance in the memory 900 are the same as each other (S530), and perform the biometric authentication of the user (S540). Meanwhile, the main processor 800 may store the first biometric information BS1 in the memory 900 (S550). That is, the main processor 800 may generate the first biometric information BS1 based on the reflected pulse wave ratio RI and perform the biometric authentication of the user. A description thereof is substantially the same as that of the embodiment of FIGS. 7 to 12, and will thus be omitted.

In the present embodiment, the main processor 800 may receive the pulse wave signal PPG and calculate the reflected pulse wave ratio RI. The main processor 800 may calculate the blood pressure based on the reflected pulse wave ratio RI in the first mode, and perform the biometric authentication based on the reflected pulse wave ratio RI in the second mode. That is, the display device 1 may calculate the blood pressure information in the first mode and perform the biometric authentication for each user without a separate biometric authentication module in the second mode by calculating the reflected pulse wave ratio RI of the pulse wave signal PPG sensed by the photo-sensor.

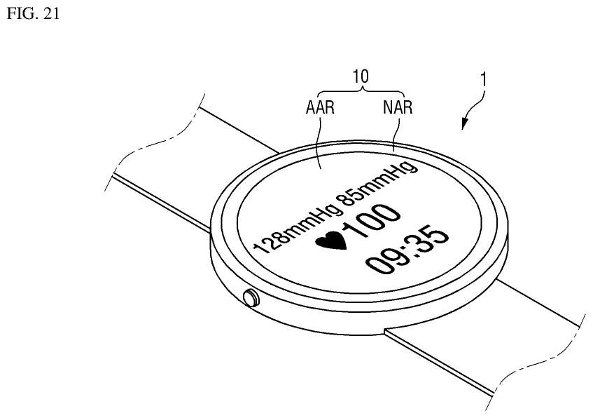

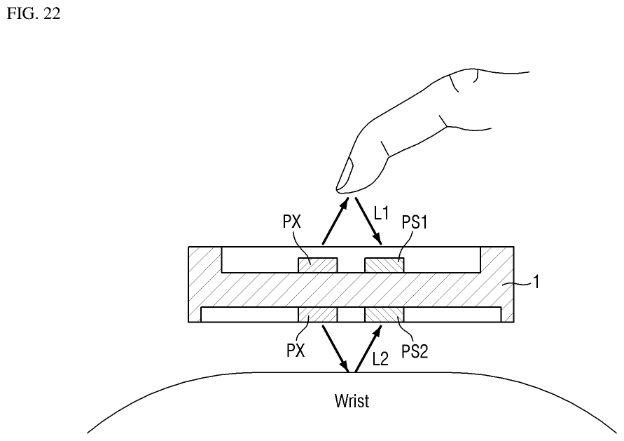

FIG. 21 is a schematic perspective view of a display device according to an embodiment of the present disclosure. FIG. 22 is a cross-sectional view of the display device of FIG. 21. The embodiment of FIGS. 21 and 22 is different from the embodiment of FIGS. 7 to 12 in that a display device includes first photo-sensors PS1 and second photo-sensors PS2. Hereinafter, contents different from those of the embodiment of FIGS. 7 to 12 will be mainly described.

Referring to FIGS. 21 and 22, a display device 1 according to an embodiment of the present disclosure includes a display panel 10. The display panel 10 serves to display a moving image or a still image. The display panel 10 may include an active area AAR and a non-active area NAR. A case where the display device 1 including the display panel 10 is a smart watch has been illustrated in FIG. 21, but the present disclosure is not limited thereto. Examples of an applicable display device 1 may include various wearable electronic devices including, for example, smart watches, portable electronic devices such as, for example, smartphones, mobile phones, tablet PCs, personal digital assistants (PDAs), portable multimedia players (PMPs), portable game machines, laptop computers, digital cameras, and camcorders, and the like. Furthermore, a case where there is a need to apply a blood pressure measurement module to fixed or mobile electronic devices including a display unit DSU, such as, for example, monitors of personal computers, vehicle navigation devices, vehicle instrument boards, external billboards, electric signs, various medical devices, various inspection devices, refrigerators, or washing machines even though they are not the portable electronic devices may be included in an application scope of embodiments. The various display devices 1 including the display panel 10, described above may be referred to as electronic devices.

Referring to FIG. 22, the display device 1 may be worn on a portion of a body of a user (or a subject). For example, the display device 1 may be configured to be worn on a wrist, an ankle, or the like.

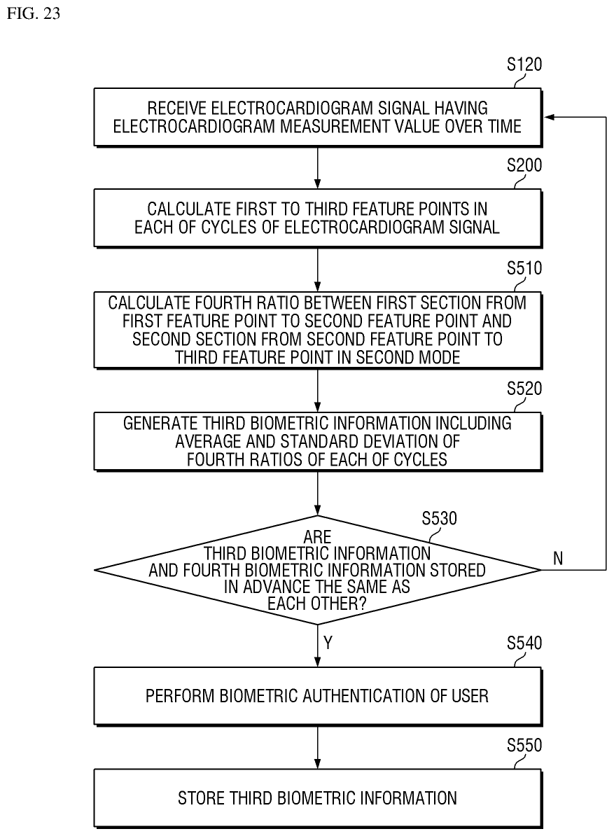

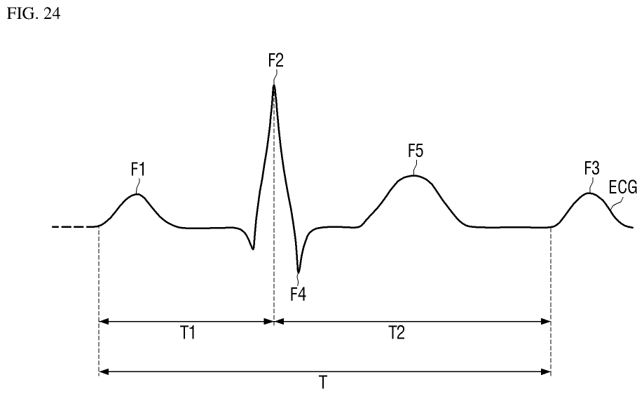

The display device 1 includes pixels PX and first photo-sensors PS1. The pixel PX and the first photo-sensor PS1 may be disposed to face upward as in the embodiment of FIG. 22. Accordingly, similar to the embodiment of FIGS. 7 to 12, the first photo-sensor PS1 may receive first light L1 obtained by reflecting light of the pixel PX by a portion (e.g., a finger) of a body of the user to measure a blood pressure. In addition, the display device 1 includes pixels PX and second photo-sensors PS2. The pixel PX and the second photo-sensor PS may be disposed to face downward as in the embodiment of FIG. 22. Accordingly, the second photo-sensor PS2 may receive second light L2 obtained by reflecting light of the pixel PX by a portion (e.g., a wrist) of the body of the user to measure an electrocardiogram. That is, in a case of the present embodiment, the first mode may be performed by the first photo-sensor PS1, and the second mode may be performed by the first photo-sensor PS1 and the second photo-sensor PS2. Accordingly, the display device 1 may measure an electrocardiogram signal (ECG of FIG. 24) to perform biometric authentication. This will be described later with reference to FIGS. 23 and 24.

FIG. 23 is a flowchart illustrating a biometric authentication method according to an embodiment of the present disclosure. FIG. 24 is a graph illustrating a waveform of an electrocardiogram signal.

The embodiment of FIGS. 23 and 24 is different from the embodiment of FIGS. 7 to 12 in that a blood pressure may be calculated in the first mode and biometric authentication may be performed in the second mode, using a reflected pulse wave ratio. Hereinafter, contents different from those of the embodiment of FIGS. 7 to 12 will be mainly described.

In the embodiment of FIGS. 23 and 24, the main processor 800 may receive a pressure signal PSS sensed by the pressure sensor, generated by the pressure sensing circuit 40 and having a pressure measurement value over time. In addition, the main processor 800 may receive a pulse wave signal PPG having a pulse wave measurement value over time, measured by the first photo-sensor PS1. A description thereof is substantially the same as that of the embodiment of FIGS. 7 to 12, and will thus be omitted.

Referring to FIG. 23, on the other hand, the main processor 800 receives an electrocardiogram signal having an electrocardiogram measurement value over time measured by the second photo-sensor PS2 (S120). Next, the main processor 800 calculates first to third feature points in each of cycles of the electrocardiogram signal (S200).

In the present embodiment, when the display device 1 includes the second photo-sensor PS2, the second photo-sensor PS2 may measure an electrocardiogram of the user by a measurement time to generate an electrocardiogram signal. The electrocardiogram signal ECG is a signal representing electrical activity of a heart at a defined time. The electrocardiogram signal ECG may be not only used to measure a rate and uniformity of a heartbeat, but may also be used to measure a size and a position of the heart, whether or not there is any damage to the heart, and the like. Accordingly, the electrocardiogram signal ECG may have a different waveform for each user, similar to the pulse wave signal PPG of the embodiment of FIGS. 7 to 12.

Referring to FIG. 24, the main processor 800 may calculate a first feature point F1 to a third feature point F3 in the electrocardiogram signal ECG. One cycle T of the electrocardiogram signal ECG may be defined as, for example, a time from the lowest point to the next lowest point. In addition, when a waveform of the electrocardiogram signal ECG is viewed in units of the cycle T, signal waveforms having a substantially similar shape may be repeated in the electrocardiogram signal ECG.

The feature points FF may be defined by inflection points of a waveform formed within one cycle T. For example, the feature points FF may include the first feature point F1 positioned at a first peak point and a third feature point F3 having the same waveform as the first feature point F1 and positioned at the next peak point, in one cycle T of the electrocardiogram signal ECG. In this case, one cycle T may be defined as a time from the first feature point F1 to the third feature point F3. In addition, the feature points FF may include the second feature point F2 positioned at the highest point in one cycle T of the electrocardiogram signal ECG. The second feature point F2 may be an upward convex peak in one cycle T of the electrocardiogram signal ECG.

The present disclosure is not limited to that described above, and the feature points FF may further include points having blood pressure information and biometric information for each user. For example, the feature points FF may further include a fourth feature point F4 positioned between the second feature point F2 and the third feature point F3 in a second section T2 and downward convex and a fifth feature point F5 positioned between the second feature point F2 and the third feature point F3 in the second section T2 and upward convex.

The main processor 800 calculates a fourth ratio between a first section T1 from the first feature point F1 to the second feature point F2 and a second section T2 from the second feature point F2 to the third feature point F3 in the second mode (S510). In addition, the main processor 800 generates third biometric information including an average and a standard deviation of the fourth ratios of each of the cycles of the electrocardiogram signal ECG (S520). A process of calculating the fourth ratio and generating the third biometric information is substantially the same as the process of calculating the first ratio based on the feature point FF and generating the first biometric information including the average and the standard deviation of the first ratios in the embodiment of FIGS. 7 to 12, and a description thereof will thus be omitted.

Next, the main processor 800 may decide whether or not the third biometric information and fourth biometric information stored in advance in the memory 900 are the same as each other (S530), and perform the biometric authentication of the user (S540). Meanwhile, the main processor 800 may store the third biometric information in the memory 900 (S550). That is, the main processor 800 may generate the third biometric information based on the electrocardiogram signal ECG and perform the biometric authentication of the user. A description thereof is substantially the same as that of the embodiment of FIGS. 7 to 12, and will thus be omitted.

In a case of the present embodiment, the main processor 800 may receive the pulse wave signal PPG, calculate the blood pressure in the first mode, and perform the biometric authentication in the second mode. That is, the display device 1 may calculate the blood pressure information in the first mode and perform the biometric authentication for each user without a separate biometric authentication module in the second mode by calculating the feature points FF of the pulse wave signal PPG sensed by the photo-sensor.

In addition to that described above, the main processor 800 may receive the electrocardiogram signal ECG and perform the biometric authentication in the second mode. That is, the display device 1 may additionally perform the biometric authentication for each user in the second mode by calculating the feature points FF of the electrocardiogram signal ECG sensed by the second photo-sensor PS2. Accordingly, the display device 1 according to the present embodiment may accurately perform the biometric authentication by using the pulse wave signal PPG and the electrocardiogram signal ECG.

In concluding the detailed description, those skilled in the art will appreciate that many variations and modifications can be made to the preferred embodiments without departing from the spirit and scope of the present disclosure as defined by the appended claims. Therefore, the disclosed preferred embodiments of the present disclosure are used in a generic and descriptive sense only and not for purposes of limitation.

What is claimed is:

1. A display device comprising:
a display panel including a plurality of pixels emitting light;
a pressure sensor sensing a pressure applied from outside;
a photo-sensor sensing light; and
a main processor receiving a pressure signal sensed by the pressure sensor and a first pulse wave signal sensed by the photo-sensor,
wherein the main processor
calculates a first feature point corresponding to a first lowest point, a second feature point corresponding to a highest point, and a third feature point corresponding to a second lowest point next to the first lowest point in each of cycles of the first pulse wave signal,
generates a peak detection signal based on an amplitude corresponding to the second feature point in each of the cycles of the first pulse wave signal and the pressure signal in a first mode and calculates blood pressure information based on the peak detection signal, and
generates first biometric information by calculating a first ratio between a first section from the first feature point to the second feature point and a second section from the second feature point to the third feature point in each of the cycles of the first pulse wave signal in a second mode and decides whether or not the first biometric information coincides with second biometric information stored in advance.

2. The display device of claim 1, wherein the main processor calculates a difference between a first pulse wave signal value corresponding to the second feature point and a first pulse wave signal value corresponding to the first lowest point as the amplitude.

3. The display device of claim 2, wherein a plurality of first ratios are calculated, and
the first biometric information includes information on an average and a standard deviation of the first ratios of each of the cycles of the first pulse wave signal.

4. The display device of claim 1, further comprising a memory in which the second biometric information is stored,
wherein the main processor is configured to store the first biometric information in the memory when the first biometric information coincides with the second biometric information stored in the memory.

5. The display device of claim 1, wherein the main processor further calculates a second ratio between an area of the first pulse wave signal in the first section and an area of the first pulse wave signal in the second section in the second mode,
a plurality of second ratios are calculated, and
the first biometric information further includes information on an average and a standard deviation of the second ratios.

6. The display device of claim 1, wherein the main processor further
generates a second pulse wave signal by calculating a second derivative graph of the first pulse wave signal over time in the second mode, and
generates the first biometric information by calculating a third ratio between a first differential value corresponding to the first feature point of the second pulse wave signal and a second differential value corresponding to the second feature point of the second pulse wave signal.

7. The display device of claim 1, wherein the main processor further calculates a peak value of the peak detection signal and a pressure value, which is referred to as PK pressure value, corresponding to the peak value of the peak detection signal in the first mode, and calculates a diastolic blood pressure lower than the PK pressure value, a systolic blood pressure higher than the PK pressure value, and a mean blood pressure according to the PK pressure value.

8. The display device of claim 7, wherein the main processor calculates the mean blood pressure as the PK pressure value corresponding to the peak value of the peak detection signal.

9. The display device of claim 7, wherein a first pressure value smaller than the PK pressure value corresponding to 60% to 80% of the peak value in the peak detection signal and a second pressure value greater than the PK pressure value are calculated, and
the first pressure value is calculated as the diastolic blood pressure and the second pressure value is calculated as the systolic blood pressure.

10. The display device of claim 1, wherein each of the cycles of the first pulse wave signal includes a plurality of waveforms having different amplitudes, and
when a peak value of a first waveform of the plurality of waveforms is defined as a pulse wave contraction value, a peak value of a second waveform of the plurality of waveforms is defined as a reflected pulse wave value, the pulse wave contraction value is defined as Sp, the reflected pulse wave value is defined as Rp, and a reflected pulse wave ratio is defined as RI, the pulse wave contraction value is the same as the amplitude corresponding to the second feature point in each of the cycles of the first pulse wave signal, and the main processor calculates the reflected pulse wave ratio by the following Equation 1.
$$R I = \frac{R p}{S p} \qquad Equation 1$$

11. The display device of claim 10, wherein the main processor
further calculates the reflected pulse wave ratio according to each of the cycles of the first pulse wave signal in the second mode, and
the first biometric information further includes the reflected pulse wave ratio.

12. The display device of claim 10, wherein the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

13. The display device of claim 12, wherein the main processor further
analyzes the reflected pulse wave ratio in the first mode to detect a start point in time of the second period,
calculates a third pressure value corresponding to the first pulse wave signal at the start point in time of the second period,
sets the third pressure value as a diastolic blood pressure,
calculates a fourth pressure value corresponding to the first pulse wave signal at a start point in time of the third period after the second period, and
sets the fourth pressure value as a systolic blood pressure.

14. A blood pressure measurement method using a display device, comprising:
calculating a first feature point corresponding to a first lowest point, a second feature point corresponding to a highest point, and a third feature point corresponding to a second lowest point next to the first feature point in each of cycles of a first pulse wave signal based on the first pulse wave signal sensed by a photo-sensor;
generating a peak detection signal based on an amplitude of the second feature point in each of the cycles of the first pulse wave signal and a pressure signal sensed by a pressure sensor in a first mode;
calculating blood pressure information based on the peak detection signal;
generating first biometric information by calculating a first ratio between a first section from the first feature point to the second feature point and a second section from the second feature point to the third feature point in each of the cycles of the first pulse wave signal in a second mode;
deciding whether or not the first biometric information coincides with second biometric information stored in advance; and
displaying the blood pressure information on a display panel in the first mode and displaying a decision result for whether or not the first biometric information coincides with the second biometric information on the display panel in the second mode.

15. The blood pressure measurement method using a display device of claim 14, wherein the amplitude of the second feature point is a difference between a first pulse wave signal value corresponding to the second feature point and a first pulse wave signal value corresponding to the first lowest point.

16. The blood pressure measurement method using a display device of claim 15, wherein the first biometric information includes information on an average and a standard deviation of ratios between the first section and the second section of each of the cycles of the first pulse wave signal.

17. The blood pressure measurement method using a display device of claim 14, wherein the generating of the first biometric information further includes:
generating a second pulse wave signal by calculating a second derivative graph of the first pulse wave signal over time; and
generating the first biometric information by calculating a third ratio between a first differential value corresponding to the first feature point of the second pulse wave signal and a second differential value corresponding to the second feature point of the second pulse wave signal.

18. The blood pressure measurement method using a display device of claim 14, wherein each of the cycles of the first pulse wave signal includes a plurality of waveforms having different amplitudes, and
when a peak value of a first waveform of the plurality of waveforms is defined as a pulse wave contraction value, a peak value of a second waveform of the plurality of waveforms is defined as a reflected pulse wave value, the pulse wave contraction value is defined as Sp, the reflected pulse wave value is defined as Rp, and a reflected pulse wave ratio is defined as RI, the pulse wave contraction value is the same as the amplitude of the second feature point in each of the cycles of the first pulse wave signal, and the reflected pulse wave ratio is calculated by the following Equation 2.
$$R I = \frac{R p}{S p} \qquad Equation 2$$

19. The blood pressure measurement method using a display device of claim 18, wherein the generating of the first biometric information further includes generating the first biometric information by calculating the reflected pulse wave ratio according to each of the cycles of the first pulse wave signal.

20. The blood pressure measurement method using a display device of claim 14, wherein in the calculating of the blood pressure information, a peak value of the peak detection signal and a pressure value, which is referred to as PK pressure value, corresponding to the peak value of the peak detection signal are calculated in the first mode, and a diastolic blood pressure lower than the PK pressure value, a systolic blood pressure higher than the PK pressure value, and a mean blood pressure according to the PK pressure value are calculated.

21. A display device comprising:
a plurality of pixels emitting light;
a pressure sensor sensing a pressure applied from outside;
a first photo-sensor disposed in the display device to face upward and sensing light;
a second photo-sensor disposed in the display device to face downward and sensing light; and
a main processor receiving a pressure signal sensed by the pressure sensor, a pulse wave signal sensed by the first photo-sensor to measure a blood pressure in a first mode, and an electrocardiogram signal sensed by the second photo-sensor,
wherein the main processor
calculates first to third feature points in each of cycles of the electrocardiogram signal, the first feature point positioned at a first peak point at a beginning of one cycle, a third feature point positioned at a second peak point at an end of the one cycle, and a second feature point positioned between the first feature point and the second feature point and at a highest point within the one cycle,
calculates a fourth ratio between a first section from the first feature point to the second feature point and a second section from the second feature point to the third feature point in a second mode,
calculates a plurality of fourth ratios and generates third biometric information including an average and a standard deviation of the fourth ratios of each of the cycles of the electrocardiogram signal,
decides whether or not the third biometric information and fourth biometric information stored in advance in a memory are the same as each other,
performs biometric authentication of a user when the third biometric information and the fourth biometric information are the same as each other, and
stores the third biometric information in the memory.
